(19) United States
(12) Patent Application Publication
(10) Pub. No.: US 2006/0134653 A1
(43) Pub. Date: Jun. 22, 2006

(54) Differential expression of genes in microsatellite instability

(75) Inventors: Christopher Burgess, Westwood, MA (US); Theodore J. Catino, Attleboro, MA (US); Gary Molino, Norfolk, MA (US); Susan Myerow, Lexington, MA (US); Arunthathi Thiagalingam, Lexington, MA (US); Marcia Lewis, Cohasset, MA (US); Stephen Thibodeau, Rochester, MN (US); Lisa Allyn Boardman, Rochester, MN (US); Lawrence Burgart, Rochester, MN (US)

Correspondence Address: SONNENSCHEIN NATH & ROSENTHAL LLP;FOR PAULA EVANS, P.O. BOX 061080, WACKER DRIVE STATION, SEARS TOWER, CHICAGO, IL, 60606-1080, US

(73) Assignees: Bayer HealthCare LLC; Mayo Foundation for Medical Education and Research

(21) Appl. No.: 11/105,233

(22) Filed: Apr. 13, 2005

Related U.S. Application Data
(60) Provisional application No. 60/591,969, filed on Jul. 28, 2004.

(51) Int. Cl. C12Q 1/68

(52) U.S. Cl. 435/6

(57) ABSTRACT

The present invention relates to nucleic acid sequences which are overexpressed in colorectal tumors which display a high level of microsatellite instability (MSI-H) vs. tumors displaying a low level of microsatellite instability (MSS), and can thus be used to identify MSI-H in a patient, and be used further to facilitate patient prognosis, monitor disease progression/regression, identify appropriate treatment regimes, and evaluate the efficacy of treatment.

### RELATED APPLICATIONS

This application claims priority under 35 U.S.C. § 119(e) to U.S. Provisional Application Ser. No. 60/591,969 which was filed on Jul. 28, 2004, the entirety of which is incorporated herein by reference.

### BACKGROUND OF THE INVENTION

Colorectal carcinoma is a malignant neoplastic disease. There is a high incidence of colorectal carcinoma in the Western world, particularly in the United States. Tumors of this type often metastasize through lymphatic and vascular channels. Many patients with colorectal carcinoma eventually die from this disease. In fact, it is estimated that 62,000 persons in the United States alone die of colorectal carcinoma annually.

However, if diagnosed early, colorectal cancer may be treated effectively by surgical removal of the cancerous tissue. Colorectal cancers originate in the colorectal epithelium and typically are not extensively vascularized (and therefore not invasive) during the early stages of development. Colorectal cancer is thought to result from the clonal expansion of a single mutant cell in the epithelial lining of the colon or rectum. The transition to a highly vascularized, invasive and ultimately metastatic cancer which spreads throughout the body commonly takes ten years or longer. If the cancer is detected prior to invasion, surgical removal of the cancerous tissue is an effective cure. However, colorectal cancer is often detected only upon manifestation of clinical symptoms, such as pain and black tarry stool. Generally, such symptoms are present only when the disease is well established, often after metastasis has occurred, and the prognosis for the patient is poor, even after surgical resection of the cancerous tissue. Early detection of colorectal cancer therefore is important in that detection may significantly reduce its morbidity.

Invasive diagnostic methods such as endoscopic examination allow for direct visual identification, removal, and biopsy of potentially cancerous growths such as polyps. Endoscopy is expensive, uncomfortable, inherently risky, and therefore not a practical tool for screening populations to identify those with colorectal cancer. Non-invasive analysis of stool samples for characteristics indicative of the presence of colorectal cancer or precancer is a preferred alternative for early diagnosis, but no known diagnostic method is available which reliably achieves this goal.

### SUMMARY OF THE INVENTION

The present invention relates to nucleic acid sequences which are overexpressed in colorectal tumors which display a high level of microsatellite instability (MSI-H) vs. tumors displaying a low level of microsatellite instability (MSS), and can thus be used to identify MSI-H in a patient, and be used further to facilitate patient prognosis, monitor disease progression/regression, identify appropriate treatment regimes, and evaluate the efficacy of treatment.

In one aspect the invention provides a method for detecting the presence of colon cancer in an subject comprising: obtaining a biological sample from said subject; and detecting the presence of the sequence of one or more of SEQ ID Nos 1-34 in said sample, wherein the presence of said one or more sequences is indicative of colon cancer in said subject

In one embodiment, the step of determining the expression level of said sequence, wherein the overexpression of said sequence by at least 2 fold relative to the expression level of said sequence in a control subject known to not have colon cancer, is indicative of the presence of colon cancer in said subject.

In a further embodiment, the step of detecting comprises contacting said biological sample with a nucleic acid probe which hybridizes under stringent hybridization conditions to one or more of said sequences, under conditions which permit stringent hybridization; and detecting the hybridization of said nucleic acid probe to said one or more of said sequences, wherein detection of hybridization is indicative of the presence of said one or more sequences in said sample.

In one embodiment, the nucleic acid probe is detectably labeled.

In a further embodiment, the subject is a human.

In another aspect, the invention also provides a method of detecting the presence of colon cancer in a subject comprising: obtaining a biological sample from said subject; and detecting the presence of one or more of the amino acid sequences of SEQ ID Nos. 35-68 in said sample, wherein the presence of said one or more sequences is indicative of colon cancer in said subject.

In one embodiment, the step of detecting comprises contacting said biological sample with a polypeptide ligand which is capable of binding to said one or more amino acid sequences under conditions which permit said polypeptide ligand to bind to said one or more amino acid sequences; and detecting the binding of said polypeptide ligand to said one or more amino acid sequences, wherein detection of binding is indicative of the presence of said one or more amino acid sequences in said sample.

In one embodiment, the polypeptide ligand is an antibody.

In a further embodiment, the polypeptide ligand comprises a detectable label.

In one embodiment, the subject is human

In another aspect, the invention also provides a method of detecting microsatellite instability in an individual comprising: measuring the expression of the sequence of one or more of SEQ ID Nos 1-34 in said individual, wherein microsatellite instability is detected where said sequence is overexpressed by at least 2-fold compared to the expression of said sequence in an individual known to not have microsatellite instability.

In one embodiment, the step of measuring comprises: obtaining a colorectal tissue sample from said individual; contacting said sample with a nucleic acid probe which is capable of hybridizing under stringent hybridization conditions to said sequence of one or more of SEQ ID Nos 1-34; detecting hybridization of said nucleic acid probe to said sequence of one or more of SEQ ID Nos 1-34.

In one embodiment, the individual is a human.

In a further embodiment, the nucleic acid probe is detectably labeled.

In another aspect, the invention also provides a method of identifying colon tissue having microsatellite instability, comprising: obtaining a colon tissue sample from an individual; detecting the expression of one or more of the sequences of SEQ ID Nos 1-34, wherein colon tissue having microsatellite instability is detected if the expression of said one or more of the sequences of SEQ ID Nos 1-34 is at least 2-fold greater than that of the same sequence in a colon tissue sample known to not have microsatellite instability.

In one embodiment, the step of detecting comprises: contacting said colon tissue sample with a nucleic acid probe which is capable of hybridizing under stringent hybridization conditions to said one or more of the sequences of SEQ ID Nos 1-34; and detecting hybridization of said nucleic acid probe to said one or more sequences, wherein detection of hybridization is indicative of the expression of said one or more of the sequences of SEQ ID Nos 1-34.

In one embodiment, the nucleic acid probe is detectably labeled.

In one embodiment, the individual is a human.

In one embodiment, the colon tissue sample is a tumor sample.

In a further embodiment, the tumor is a malignant tumor.

In another aspect, the invention also provides a method of monitoring the onset, progression, or regression of cancer or a pre-malignant condition thereof in a subject, the method comprising: detecting in a biological sample of the subject at a first point in time, the expression of one or more nucleic acid sequences comprising one or more nucleic acid sequences selected from the group consisting of SEQ ID NOs: 1-34; repeating step (a) at a subsequent point in time; and comparing the expression level detected in steps (a) and (b), wherein a change in the expression level is indicative of progression of cancer or a pre- malignant condition thereof in the subject.

In one embodiment, the change in the expression level is either an increase or decrease.

In a further embodiment, the increase or decrease is an increase or decrease of at least 2 fold.

In another aspect, the present invention also provides a method of determining prognosis for cancer or a pre-malignant condition thereof in a subject, comprising: detecting in a biological sample of the subject, the expression level of one or more nucleic acid sequences comprising one or more nucleic acid sequences selected from the group consisting of SEQ ID NOs: 1-34; comparing the expression level detected in steps (a) with a reference expression level of said nucleic acid sequences; and evaluating the prognosis of the subject based on the comparison in step (b).

In one embodiment, the reference expression level is the expression level of said nucleic acid sequences in cancer free or normal sample.

In a further embodiment, the reference expression level is the expression level of said nucleic acid sequences in cancer samples that are known not to progress to aggressive form.

In one embodiment, the reference expression level is the expression level of said nucleic acid sequence in a MSS colon tumor.

In another aspect, the invention also provides a method of determining the efficacy of a test compound for inhibiting cancer in a subject, the method comprising comparing a) the expression level of one or more nucleic acid sequences in a first biological sample from the subject wherein the sample has been exposed to the test compound, with b) the expression level of said nucleic acid sequences in a second biological sample from the subject wherein the sample has not been exposed to the test compound, said nucleic acid sequences comprising one or more nucleic acid sequences selected from the group consisting of SEQ ID NOs: 1-34, wherein a change of at least two fold in the expression level of said nucleic acid sequences is an indication that the test compound is efficacious for inhibiting cancer in the subject.

In one embodiment, the change in the expression level is either an increase or decrease.

In a further aspect, the invention also provides a method of determining the efficacy of a therapy for inhibiting cancer in a subject, the method comprising comparing a) the expression level of one or more nucleic acid sequences in a first biological sample from the subject prior to providing at least a portion of the therapy to the subject, with b) the expression level of said nucleic acid sequences in a second biological sample from the subject following the provision of at least a portion of the therapy, said nucleic acid sequences comprising one or more nucleic acid sequences selected from the group consisting of SEQ ID NOs: 1-34, wherein a change of at least two fold in the expression level of said nucleic acid sequences is an indication that the therapy is efficacious for inhibiting cancer in the subject.

In one embodiment, the change in the expression level is either an increase or decrease.

In another aspect, the invention also provides a method of selecting a composition for inhibiting cancer in a subject, the method comprising: obtaining a first biological sample comprising cancer cells from the subject; separately exposing aliquots of the sample in the presence of a plurality of test compositions; comparing the expression level of one or more nucleic acid sequences in each of the aliquots from (b) with the expression level in the sample produced by (a), said nucleic acid sequences comprising one or more nucleic acid sequences selected from the group consisting of SEQ ID NOs: 1-34; and selecting one of the test compositions which induces a change of at least two fold in the expression level of said nucleic acid sequences in one aliquot containing the test composition.

In one embodiment, the change in the expression level is either an increase or decrease.

In another aspect, the invention also provides A method of monitoring the onset, progression, or regression of cancer in a subject, comprising: contacting at a first point in time a first biological sample with one or more polypeptide ligands that specifically bind to one or more polypeptides comprising a polypeptide sequence selected from the group consisting of SEQ ID NOs: 35-68, determining specific binding between the polypeptide ligands and the polypeptides; contacting at a subsequent point in time a second biological sample with said polypeptide ligands that specifically bind to one or more polypeptides comprising a polypeptide sequence selected from the group consisting of SEQ ID NOs: 35-68, determining specific binding between the polypeptide ligands and the polypeptides; and comparing the specific binding in the first biological sample to the specific binding in the second biological sample, wherein a significant change in the specific binding is an indication of the onset, progression, or regression of cancer.

In another aspect, the invention also provides a method of determining prognosis for cancer or a pre-malignant condition thereof in a subject, comprising: contacting a biological sample obtained from a subject having cancer with one or more polypeptide ligands that bind specifically to one or more polypeptides comprising a polypeptide sequence selected from the group consisting of SEQ ID NOs: 35-68; determining specific binding; comparing the specific binding between the polypeptide ligands and the polypeptides in the sample with the specific binding between the polypeptide ligands and the polypeptides either in a cancer-free sample or in a cancer sample that is known not to progress to aggressive form; and evaluating the prognosis of the subject based on the comparison in step (c).

In a still further aspect, the invention also provides a method of determining the efficacy of a test compound for inhibiting cancer in a subject, the method comprising comparing a) in a first biological sample from the subject binding between one or more polypeptide ligands that specifically bind to one or more polypeptides comprising a polypeptide sequence selected from the group consisting of SEQ ID NOs: 35-68 and one or more polypeptides comprising a polypeptide sequence selected from the group consisting of SEQ ID NOs: 35-68, wherein the sample has not been exposed to the test compound, with b) in a second biological sample from the subject, the specific binding of said polypeptide ligands and said polypeptides, wherein the sample has been exposed to the test compound, and wherein a significant change in the specific binding is an indication that the test compound is efficacious for inhibiting cancer in the subject.

In another aspect, the invention provides a method of determining the efficacy of a therapy for inhibiting cancer in a subject, comprising comparing a) in a first biological sample from the subject prior to a treatment, binding between one or more polypeptide ligands that specifically bind to one or more polypeptides comprising a polypeptide sequence selected from the group consisting of SEQ ID NOs: 35-68 and one or more polypeptides comprising a polypeptide sequence selected from the group consisting of SEQ ID NOs: 35-68, with b) in a second biological sample from the subject following the treatment, the specific binding of said polypeptide ligands and said polypeptides, and wherein a significant change in the specific binding is an indication that the test compound is efficacious for inhibiting cancer in the subject.

### BRIEF DESCRIPTION OF THE DRAWINGS

FIG. 1 shows the differential expression profile of the MSI-H marker gene REG4.

### DETAILED DESCRIPTION

Two primary mechanisms of genomic instability have been discovered to play a role in sporadic colorectal cancer. The first is known as chromosomal instability, and results from a series of genetic changes that involve the activation of a set of oncogenes such as ki-ras and inactivation of tumor suppressor genes such as p53 and APC. Cells displaying chromosomal instability exhibit large-scale chromosomal alterations such as loss of heterozygosity, affecting hundreds of thousands of DNA bases. The second, mechanism, known as microsatellite instability (MSI), was originally described in hereditary non-polyposis colon cancer, and is also found in 12-15% of sporadic colorectal cancers. The indications of MSI is the widespread mutation of short repetitive DNA sequences known as microsatellites. MSI tumors have a distinct phenotype, characterized by mucinous histology, prominent lymphocytic infiltration, proximal location (proximal to the splenic flexure), poor differentiation and, more favorable stage distribution (i.e., is identified at earlier stages of cancer progression) (See, e.g., Gervaz et al., 2003. Swiss Surg. 9:3). There is a need in the art for a method for rapid, and accurate detection of MSI tumors, and for reliable criteria for the determination of prognosis and treatment regime for patients with MSI colorectal tumors.

The present invention is based, in part, on the discovery that certain gene sequences are overexpressed in human colorectal tumors that are classified as microsatellite unstable (MSI-H; classification is described further below) relative to those tumors which do not show microsatellite instability (MSS). More specifically, the present invention relates to 34 gene sequences which are overexpressed by at least 2-fold in colorectal tumors with a high degree of microsatellite instability (MSI-H) relative to colorectal tumors which do not demonstrate microsatellite instability. Accordingly, in one embodiment, the discovery of gene overexpression in MSI-H colorectal tumors provides a means for the detection and prediction of colon cancer in an individual. The present invention provides a method whereby a biological sample may be obtained from an individual suspected of having colon cancer, and subsequently screened, using nucleic acid probes, to determine whether the one or more of the gene sequences described herein are overexpressed by at least 2-fold. Identification of at least 2-fold overexpression of one or more of the gene sequences of the invention permits one of skill in the art to classify the individual as having a colorectal cancer which is MSI-H vs. MSI-L vs. MSS, which provides the practitioner with valuable prognostic information for the individual. In a preferred embodiment, the present invention provides a method for determining the prognosis of, course of treatment for, efficacy of treatment for, and monitoring of progression and/or regression and/or recurrence of colorectal cancer in a patient, based on the overexpression of one or more of the MSI-H marker sequences of the invention.

Accordingly, the invention provides MSI specific markers (nucleic acid sequences: SEQ ID Nos 1-34; amino acid sequences: 35-68) which are useful for the detection of colorectal cancer, and microsatellite instability. The MSI-H markers may also be used according to the invention for the prediction of the occurrence of colorectal cancer, and for determining an individual's predisposition to the development of colorectal cancer.

Definitions

As used herein, “microsatellite instability” (MSI) refers to the expansion or contraction of short nucleotide repeats (microsatellites) within a tumor when compared to normal tissue. MSI does not describe a particular tumor phenotype but refers instead to the observation of instability at one or more given genetic marker. A microsatellite is DNA in which a short motif(usually one to five nucleotides long) is repeated several times. A typical mononucleotide-repeat microsatellite might be, for example, a stretch of 13 adenines, abbreviated (A)₁₃. The most common microsatellite in humans is a dinucleotide repeat of cytosine and adenine, (CA)ₙ, which occurs in tens of thousands of locations in our germ line. The microsattelite repeats are prone to expansion or contractions (lengthening or shortening) in meiosis. While these expansions or contractions are normally repaired by DNA mismatch repair proteins (MMR), defects in MMRs can result in the persistence of such microsatellite modification, which is then referred to as microsatellite instability. MSI is subdivided into three categories: MSI-H, MSI-L and MSS (microsatellite stable). MSI-H, MSI-L and MSS tumors are defined as described in Boland et al. (1998, Cancer Research 58:5248; discussed in further detail below).

As used herein, a “biological sample” refers to a tissue, cellular, or fluid sample obtained from an individual. A “biological sample”, as used herein, can refer to a cells, circulating cells (e.g., circulating cells in blood), cells obtained from specific anatomical locations, or specific cell types (e.g., colon cell, gastrointestinal cell, cancerous cell, etc.), a tissue sample, or physiological fluids such as lymph, bile, serum, plasma, urine, synovial fluid, blood, CSF, mucus membrane secretions, or other physiological samples such as stool. Preferably, the biological sample is colon tissue. An MSI associated marker of the invention, such as one or more of SEQ ID Nos 1-34, may be detected in a suitable “biological sample” where the suitability of a particular type of biological sample for the detection of a specific colorectal cancer associated marker may be readily determined by one of skill in the art.

As used herein, “detecting” refers to the identification of the presence or absence of a molecule in a sample. Where the molecule to be detected is a polypeptide, the step of detecting can be performed by binding the polypeptide with a peptide ligand, such as an antibody, that is detectably labeled. A detectable label is a molecule which is capable of generating, either independently, or in response to a stimulus, an observable signal. A detectable label can be, but is not limited to a fluorescent label, a chromogenic label, a luminescent label, or a radioactive label. Methods for “detecting” a label include quantitative and qualitative methods adapted for standard or confocal microscopy, FACS analysis, and those adapted for high throughput methods involving multiwell plates, arrays or microarrays. One of skill in the art can select appropriate filter sets and excitation energy sources for the detection of fluorescent emission from a given fluorescent polypeptide or dye. “Detecting” as used herein can also include the use of multiple peptide ligands to a polypeptide to be detected, wherein the multiple antibodies bind to different epitopes on the polypeptide to be detected. Antibodies used in this manner can employ two or more detectable labels, and can include, for example a FRET pair. A polypeptide molecule, such as one or more of SEQ ID Nos 35-68, is “detected” according to the present invention when the level of detectable signal is at all greater than the background level of the detectable label, or where the level of measured nucleic acid is at all greater than the level measured in a control sample.

As used herein, “detecting” as it refers to detecting the presence of a target nucleic acid molecule (e.g., a nucleic acid molecule of one or more of SEQ ID Nos 1-34) refers to a process wherein the signal generated by a directly or indirectly labeled probe nucleic acid molecule (capable of hybridizing to a target, e.g., a sequence of one or more of SEQ ID Nos 1-34 in a biological sample; e.g., in a cell by in situ hybridization) is measured or observed. Thus, detection of the probe nucleic acid is directly indicative of the presence, and thus the detection, of a target nucleic acid. For example, if the detectable label on the probe nucleic acid is a fluorescent label, the target nucleic acid is “detected” by observing or measuring the light emitted by the fluorescent label on the probe nucleic acid when it is excited by the appropriate wavelength, or if the detectable label is a fluorescence/quencher pair, the target nucleic acid is “detected” by observing or measuring the light emitted upon association or dissociation of the fluorescence/quencher pair present on the probe nucleic acid, wherein detection of the probe nucleic acid indicates detection of the target nucleic acid. If the detectable label is a radioactive label, the target nucleic acid, following hybridization with a radioactively labeled probe is “detected” by, for example, autoradiography. Methods and techniques for “detecting” fluorescent, radioactive, and other chemical labels may be found in Ausubel et al. (1995, Short Protocols in Molecular Biology, 3^{rd} Ed. John Wiley and Sons, Inc.). Alternatively, a nucleic acid may be “indirectly detected” wherein a moiety, such as an enzyme activity, is attached to a probe nucleic acid which will hybridize with the target allowing detection in the presence of an appropriate substrate, or a specific antigen or other marker allowing detection by addition of an antibody or other specific indicator. Alternatively, a target nucleic acid molecule can be detected by amplifying a nucleic acid sample prepared from a biological sample, using oligonucleotide primers which are specifically designed to hybridize with a portion of the target nucleic acid sequence. Quantative amplification methods, such as, but not limited to TaqMan, may also be used to “detect” a target nucleic acid according to the invention. A nucleic acid molecule is “detected” as used herein where the level of nucleic acid measured (such as by quantitative PCR), or the level of detectable signal provided by the detectable label is at all above the background or control level.

As used herein, “detecting” also refers further to the detection of colorectal cancer in a patient, preferably early detection, wherein “early” detection refers to the detection of colorectal cancer at Dukes stage A or preferably, prior to a time when the colorectal cancer is morphologically able to be classified in a particular Dukes stage. “Detecting” as used herein further refers to the detection of colorectal cancer recurrence in an individual, using the same detection criteria as indicated above. “Detecting” as used herein still further refers to the measuring of a change in the degree of colorectal cancer before and/or after treatment with a therapeutic agent. In this case, a change in the degree of colorectal cancer in response to a therapeutic agent refers to an increase or decrease in the expression of one or more of the MSI-H markers of the invention, or alternatively, in the amount of MSI-H marker polypeptide (e.g., one or more of SEQ ID Nos 35-68) present in a clinical sample by at least 10% in response to the presence of a therapeutic agent relative to the expression level in the absence of the therapeutic agent.

As used herein, the term “change in the expression level” refers to either an increase or a decrease of the expression level of a nucleotide sequence (e.g., measuring the level of nucleic acid expressed and/or measuring the level of protein produced) in a test sample from the control level by an amount greater than the standard error of the assay employed to assess expression. Preferably, the change is by at least about twice, and more preferably three, four, five or ten times that amount. For increase, the change is determined by comparing the expression level in the test sample to the control level. For decrease, the change is determined by comparing the control level to the expression level in the test sample. Alternatively, the decrease is determined by comparing the expression level in the test sample to the control level and the decrease in the expression level is by at least about 15%, 25%, 30%, 40%, 50%, 65%, 80%, or greater. The term “significant change in the specific binding” refers to either an increase or a decrease from the specific binding in the cancer-free sample by at least about 10%, 20%, 25%, 30%, preferably at least about 40%, 50%, more preferably at least about 60%, 70%, or 90%.

As used herein, the term “expression level of one or more nucleic acid sequences” refers to the amount of mRNA transcribed from the corresponding genes (or the amount of DNA transcribed to mRNA) that are present in a biological sample. The expression level can be detected with or without comparison to a level from a control sample or a level expected of a control sample.

As used herein, the term “control expression level of one or more nucleic acid sequences” refers to the amount of mRNA transcribed from the corresponding genes (or the amount of DNA transcribed to mRNA) that are present in a biological sample representative of healthy, cancer-free subjects. The term “control expression level” can also refer to an established level of mRNA representative of the cancer-free population, that has been previously established based on measurement from healthy, cancer-free subjects.

As used herein, an “MSI-H marker sequence” refers to the nucleic acid sequences of SEQ ID Nos 1-34 which encode the amino acid sequences of SEQ ID Nos 35-68, and which have been identified according to the present invention as being overexpressed in colorectal tumor tissue having microsatellite instability. MSI-H marker sequences according to the invention include both the nucleic acid sequences of SEQ ID Nos 1-34, and the amino acid sequences of SEQ ID Nos 35-68. The MSI-H marker sequences of the invention provide useful tools for the detection of colorectal cancer, the identification of microsatellite instability in a biological sample, and prediction of the development of colon cancer.

As used herein, the term “cancerous cell” or “cancer cell”, used either in the singular or plural form, refers to cells that have undergone a malignant transformation that makes them pathological to the host organism. Malignant transformation is a single- or multi-step process, which involves in part an alteration in the genetic makeup of the cell and/or the gene expression profile. Malignant transformation may occur either spontaneously, or via an event or combination of events such as drug or chemical treatment, radiation, fusion with other cells, viral infection, or activation or inactivation of particular genes. Malignant transformation may occur in vivo or in vitro, and can if necessary be experimentally induced. Malignant cells may be found within the well-defined tumor mass or may have metastasized to other physical locations. A feature of cancer cells is the tendency to grow in a manner that is uncontrollable by the host, but the pathology associated with a particular cancer cell may take any form. Primary cancer cells (that is, cells obtained from near the site of malignant transformation) can be readily distinguished from non-cancerous cells by well-established pathology techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells.

As used herein, the term “efficacy” refers to either inhibition to some extent, of cell growth causing or contributing to a cell proliferative disorder, or the inhibition, to some extent, of the production of factors (e.g., growth factors) causing or contributing to a cell proliferative disorder. A “therapeutic efficacy” refers to relief of one or more of the symptoms of a cell proliferative disorder. In reference to the treatment of a cancer, a therapeutic efficacy refers to one or more of the following: 1) reduction in the number of cancer cells; 2) reduction in tumor size; 3) inhibition (i.e., slowing to some extent, preferably stopping) of cancer cell infiltration into peripheral organs; 3) inhibition (i.e., slowing to some extent, preferably stopping) of tumor metastasis; 4) inhibition, to some extent, of tumor growth; and/or 5) relieving to some extent one or more of the symptoms associated with the disorder. In reference to the treatment of a cell proliferative disorder other than a cancer, a therapeutic efficacy refers to 1) either inhibition to some extent, of the growth of cells causing the disorder; 2) the inhibition, to some extent, of the production of factors (e.g., growth factors) causing the disorder; and/or 3) relieving to some extent one or more of the symptoms associated with the disorder.

As used herein, the term “detectable label” refers to a composition detectable by spectroscopic, photochemical, biochemical, immunochemical, or chemical means.

As used herein, the term “a polynucleotide probe” refers to a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified on bases (7-deazaguanosine, inosine, etc.) or on sugar moiety. In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

As used herein, the term “hybridization” refers to any process by which a strand of nucleic acid binds with a complementary strand through base pairing.

As used herein, the term “subject” refers to any human or non-human organism.

As used herein, “individual” refers to a mammal, preferably a human.

As used herein, a “ligand” refers to a molecule which is capable of binding a polypeptide or nucleic acid. A “polypeptide ligand” useful in the present invention includes, but is not limited to an antibody, a monoclonal antibody, a polyclonal antibody, an antibody fragment (e.g., Fv, scFV, or Fab), a small molecule, or a nucleic acid aptamer. A “ligand” as used herein can also refer to a “nucleic acid ligand”, such as an oligonucleotide, polynucleotide, DNA, RNA, mRNA, or cDNA, which is capable of binding to a complementary nucleic acid molecule, or polypeptide molecule.

The term “antibody” as used herein is intended to include whole antibodies, e.g., of any isotype (IgG, IgA, IgM, IgE, etc), and includes fragments thereof, and single-chain antibodies, which also are specifically reactive with a vertebrate, e.g., mammalian, protein. Antibodies can be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, or other purified preparations of antibodies and recombinant antibodies.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

The terms “protein”, “polypeptide”, and “peptide” are used interchangeably herein when referring to a gene product. As used herein, “polypeptide” refers to any kind of polypeptide such as peptides, human proteins, fragments of human proteins, proteins or fragments of proteins from non-human sources, engineered versions proteins or fragments of proteins, enzymes, antigens, drugs, molecules involved in cell signaling, such as receptor molecules, antibodies, including polypeptides of the immunoglobulin superfamily, such as antibody polypeptides or T-cell receptor polypeptides.

As used herein, the term “level of expression” or “expression level” refers to the measurable expression of a given nucleic acid. The level of expression of a nucleic acid is determined by methods well known in the art. The “level of expression” or an “expression level” may measured by hybridization analysis using labeled target nucleic acids according to methods well known in the art (see, for example, Ausubel et al., Short Protocols in Molecular Biology, 3^{rd} Ed. 1995, John Wiley and Sons, Inc.). The label on the target nucleic acid is a luminescent label, an enzymatic label, a radioactive label, a chemical label or a physical label. Preferably, the target nucleic acids are labeled with a fluorescent molecule. Preferred fluorescent labels include fluorescein, amino coumarin acetic acid, tetramethylrhodamine isothiocyanate (TRITC), Texas Red, Cy3 and Cy5. Alternatively, the “level of expression” can be measured by quantitative amplification protocols, such as TaqMan, known to those of skill in the art.

“Differential expression” as used herein refers to an increase or decrease in expression of a nucleic acid sequence of at least about 2 fold, at least about 3 fold, at least about 10 fold, or at least up to or greater than about 50 fold. Similarly, “overexpression” refers to an increase in the expression of a nucleic acid sequence of at least about 2 fold, at least about 3 fold, at least about 10 fold, or at least up to or greater than about 50 fold. In a preferred embodiment a sequence of one or more of SEQ ID Nos 1-34 is overexpressed if it is over expressed by an average of at least about 2 fold as measured in at least two individuals with colon cancer relative to the average expression level in at least two normal individuals, or alternatively, as measured in a colon cancer sample from an individual relative to an adjacent, healthy colon tissue sample. The method of determining the phenotype of a cell (e.g., detecting colon cancer or microsatellite instability) may comprise detecting the differential expression, relative to a normal cell, of at least one nucleic acid which hybridizes under stringent conditions one of SEQ ID Nos. 1-34, wherein the nucleic acid is differentially expressed by at least at least about 2 fold, at least about 3 fold, at least about 10 fold, or at least up to or greater than about 50 fold. Alternatively, the method of determining the phenotype of a cell may comprise detecting the differential expression, relative to a normal cell, of at least one nucleic acid comprising SEQ ID Nos 1-34.

As used herein, “stringent hybridization conditions” refers to conditions of temperature and buffer composition which permit hybridization of a first nucleic acid sequence to a second nucleic acid sequence, and preferably refers to either high or moderate stringency hybridization conditions. Therefore, “high stringency conditions” are those conditions wherein only nucleic acid sequences which are very similar to each other will hybridize. The sequences may be less similar to each other if they hybridize under moderate stringency conditions. The precise conditions determining the stringency of a particular hybridization include not only the ionic strength, temperature, and the concentration of destabilizing agents such as formamide, but also on factors such as the length of the nucleic acid sequences, their base composition, the percent of mismatched base pairs between the two sequences, and the frequency of occurrence of subsets of the sequences (e.g., small stretches of repeats) within other non-identical sequences. Washing is the step in which conditions are set so as to determine a minimum level of similarity between the sequences hybridizing with each other. Generally, from the lowest temperature at which only homologous hybridization occurs, a 1% mismatch between two sequences results in a 1° C. decrease in the melting temperature (Tₘ) for any chosen SSC concentration. Generally, a doubling of the concentration of SSC results in an increase in the Tₘ of about 17° C. Using these guidelines, the washing temperature can be determined empirically, depending on the level of mismatch sought. Hybridization and wash conditions are explained in Current Protocols in Molecular Biology (Ausubel, F. M. et al., eds., John Wiley & Sons, Inc., 1995, with supplemental updates) on pages 2.10.1 to 2.10.16, and 6.3.1 to 6.3.6.

“High stringency conditions”, as used herein, generally refers to hybridization at either (1) 1×SSC (10×SSC=3 M NaCl, 0.3 M Na₃-citrate.2H₂O (88 g/liter), pH to 7.0 with 1 M HCl), 1% SDS (sodium dodecyl sulfate), 0.1-2 mg/ml denatured salmon sperm DNA at 65° C.; (2) 1×SSC, 50% formamide, 1% SDS, 0.1-2 mg/ml denatured salmon sperm DNA at 42° C.; (3) 1% bovine serum albumen (fraction V), 1 mM Na₂.EDTA, 0.5 M NaHPO₄ (pH 7.2) (1 M NaHPO₄=134 g Na₂HPO₄.7H₂O, 4 ml 85% H₃PO₄ per liter), 7% SDS, 0.1-2 mg/ml denatured salmon sperm DNA at 65° C.; (4) 50% formamide, 5×SSC, 0.02 M Tris-HCl (pH 7.6), 1× Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 42° C.; (5) 5×SSC, 5× Denhardt's solution, 1% SDS, 100:g/ml denatured salmon sperm DNA at 65° C.; or (6) 5×SSC, 5× Denhardt's solution, 50% formamide, 1% SDS, 100:g/ml denatured salmon sperm DNA at 42° C., with high stringency washes of either (1) 0.3-0.1×SSC, 0.1% SDS at 65° C.; or (2) 1 mM Na₂EDTA, 40 mM NaHPO₄ (pH 7.2), 1% SDS at 65° C. The above conditions are intended to be used for DNA- DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2× the number of A and T bases)+(4× the number of G and C bases). For hybrids believed to be about 18 to about 49 base pairs in length, the Tₘ in ° C.=(81.5° C.+16.6(log₁₀M)+0.41(% G+C)−0.61 (% formamide)-500/L), where “M” is the molarity of monovalent cations (e.g., Na⁺), and “L” is the length of the hybrid in base pairs.

“Moderate stringency conditions” refers to hybridization at either (1) 4×SSC, (10×SSC=3 M NaCl, 0.3 M Na₃-citrate.2H₂O (88 g/liter), pH to 7.0 with 1 M HCl), 1% SDS (sodium dodecyl sulfate), 0.1-2 mg/ml denatured salmon sperm DNA at 65° C.; (2) 4×SSC, 50% formamide, 1% SDS, 0.1-2 mg/ml denatured salmon sperm DNA at 42° C.; (3) 1% bovine serum albumen (fraction V), 1 mM Na₂.EDTA, 0.5 M NaHPO₄ (pH 7.2) (1 M NaHPO₄=134 g Na₂HPO₄.7H₂O, 4 ml 85% H₃PO₄ per liter), 7% SDS, 0.1-2 mg/ml denatured salmon sperm DNA at 65° C.; (4) 50% formamide, 5×SSC, 0.02 M Tris-HCl (pH 7.6), 1× Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 42° C.; (5) 5×SSC, 5× Denhardt's solution, 1% SDS, 100 μg/ml denatured salmon sperm DNA at 65° C.; or (6) 5×SSC, 5× Denhardt's solution, 50% formamide, 1% SDS, 100:g/ml denatured salmon sperm DNA at 42° C., with moderate stringency washes of 1×SSC, 0.1% SDS at 65° C. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2× the number of A and T bases)+(4× the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

As used herein, the terms specific “binding” or “specifically binding”, refers to the interaction of an antibody and a protein or peptide. The interaction is dependent upon the presence of a particular structure (i.e., the antigenic determinant or epitope) on the protein; in other words, the antibody is recognizing and binding to a specific protein structure rather than to proteins in general. For example, if an antibody is specific for epitope A, the presence of a protein containing epitope A (or free, unlabeled A) in a reaction containing labeled “A” and the antibody will reduce the amount of labeled A bound to the antibody.

As used herein, a “predisposition to developing colon cancer” refers to a condition under which any given population of individuals who are “predisposed to developing colon cancer” will have an at least 10% increase in the incidence of colon cancer than a similar population not predisposed to developing colon cancer. As used herein, a “predisposition to developing colon cancer” also refers to a condition in an individual wherein the expression of one or more of the MSI-H marker sequences of the invention is identified in colon tissue of the individual as being overexpressed by at least 2-fold relative to the expression level in a normal individual.

MSI-H Marker Sequences

Microsatellite Instability

Microsatellite instability refers to the expansion or contraction of short nucleotide repeats (microsatellites) within a tumor when compared to normal tissue. MSI does not describe a particular tumor phenotype but refers instead to the observation of instability at a given genetic marker. MSI is divided into three subcategories. The first is characterized by MSI-H, wherein a majority of markers exhibit MSI; the second is MSI-L, wherein only a minority of markers exhibit MSI; and the third is MSS, wherein markers lack apparent instability. The MSI status of a given tumor is determined by looking at microsatellite instability in a panel of genetic markers, comprising two markers which include mononucleotide repeats (BAT26 and BAT25, GenBank Accession Nos. 9834508 and 9834505, respectively), and three markers having dinucleotide repeats (D5S346, D2S123, and D17S250, GenBank Accession Nos 181171, 187953, and 177030, respectively). Using this reference panel, MSI-H tumors are defined as having instability in two or more markers, while MSI-L tumors are defined as having instability in one marker. Tumors showing no apparent instability may be included in the MSS group. In addition to the five primary markers described above, additional markers may be examined for microsatellite instability including, but not limited to, BAT40, BAT34C4, TGF-β-R11, ACTC(635/636), D18S55, D18S58, D18S61, D18S64, D3S1029, D10S197, D13S175, D17S588, D5S107, D8S87, D18S69, D13S153, D17S787, D7S519, and D20S100. Where more than the primary panel of five markers is used, MSI-H is defined as having MSI in ≧30-40% of the markers tested, whereas the MSI-L group would exhibit MSI in <30-40% of the markers. A description of the criteria for characterizing MSI-H vs. MSI-L vs. MSS may be found, for example, in Boland et al., 1998, Cancer Research 58:5248, the contents of which are incorporated herein by reference.

The distinction between MSI-H and MSI-L/MSS tumors is important for the prognosis, treatment and monitoring of colorectal cancers. For example, MSI status (that is, the classification of a tumor as MSI-H, MSI-L, or MSS) has been shown to be a predictor of the benefit of adjuvant-based chemotherapy with fluorouracil in stage II and stage III colon cancers (Ribic et al., 2003 N. Engl. J. Med. 349:247). In addition, the proximal anatomical localization of MSI-H tumors has been linked to a more favorable outcome in patients (e.g., higher rate of survival; Boland et al.; Gervaz et al., 2003, Swiss Surg. 9:3; and Rubic et al.), and MSI-H tumors have been correlated to a more favorable stage distribution, with the majority of MSI-H tumors being grouped in Stage II colorectal cancers (Gervaz et al). Thus, as described further below, the identification and classification of colorectal tumors into MSI status provides the medical practitioner with critical information as to patient diagnosis, prognosis, and optimal treatment regime.

Traditionally, the MSI status of colorectal cancer tumors has been detected by genetic screens for the markers described above. This method of MSI staging is not trivial and is cumbersome and time consuming, requiring comparisons of normal to tumor DNA bands which have been separated out by, for example, polyacrylamide gel electrophoresis or other suitable method (see, for example, Bapat et al, 1999, Human Genetics 104:167). It is also generally accepted that the genetic detection of MSI using the marker panels described above should also include microdissection of the tumor tissue to enrich for tumor cell populations (see, e.g., Boland et al., Id.). In contrast, the present invention provides a means for determining the MSI status of a tumor by detecting the overexpression of one or more of the MSI-H marker genes described herein. The present invention thus, provides an alternative to the genetic (i.e., marker panel) approach for MSI detection which is used in the art to date, and is useful for determining the prognosis, staging, and treatment regime for a patient having a colorectal cancer.

The present invention related to the detection of colon cancer and microsatellite instability by detecting the overexpression of one or more of the MSI-H marker sequences disclosed herein. The present invention equally relates to the marker sequences themselves, which have been identified as being overexpressed by at least 2-fold in MSI-H colorectal tumors relative to normal colon tissue.

In addition the invention encompasses the detection of cancer by identifying the one or more of polypeptide sequences encoded by the MSI-H marker sequences in colon tissue or cells. Alternatively, the invention relates to a method for the detection of colorectal cancer in an individual wherein colorectal cancer is identified by detecting the overexpression of one or more MSI-H marker nucleic acid sequences and at least one additional colorectal cancer associated marker in the biological sample from an individual. MSI-H marker sequences of the invention include those having the nucleic acid sequence shown in one or more of SEQ ID Nos. 1-34, or alternatively, the polypeptide sequences encoded by the recited polynucleotide sequences as shown in SEQ ID Nos. 34-68.

The nucleic acid and amino acid sequences of the MSI-H markers are shown in SEQ ID Nos 1-34 and 35-68, respectively (as shown in Table 1 and the sequence listing). While the invention relates to the direct detection of the MSI-H marker sequences in a method for detecting colorectal cancer or MSI, the invention further relates to the detection of sequences complementary thereto, or a sequence which specifically hybridizes to a sequence of SEQ ID Nos. 1-34. The present invention also relates to the detection of colorectal cancer or MSI by detecting the presence, in a clinical sample, of a nucleic acid molecule which encodes the sequence of SEQ ID Nos. 35-68, or a fragment thereof.

Another aspect of the invention provides the detection of MSI-H by the detection of a nucleic acid which hybridizes under low, medium, or high stringency, preferably medium or high, conditions to a nucleic acid sequence represented by one or more of SEQ ID Nos. 1-34, or a sequence complementary thereto. Stringency conditions for hybridization refers to conditions of temperature and buffer composition which permit hybridization of a first nucleic acid sequence to a second nucleic acid sequence, wherein the conditions determine the degree of identity between those sequences which hybridize to each other. Therefore, “high stringency conditions” are those conditions wherein only nucleic acid sequences which are very similar to each other will hybridize. The sequences may be less similar to each other if they hybridize under moderate stringency conditions. Still less similarity is needed for two sequences to hybridize under low stringency conditions. By varying the hybridization conditions from a stringency level at which no hybridization occurs, to a level at which hybridization is first observed, conditions can be determined at which a given sequence will hybridize to those sequences that are most similar to it. The precise conditions determining the stringency of a particular hybridization include not only the ionic strength, temperature, and the concentration of destabilizing agents such as formamide, but also on factors such as the length of the nucleic acid sequences, their base composition, the percent of mismatched base pairs between the two sequences, and the frequency of occurrence of subsets of the sequences (e.g., small stretches of repeats) within other non-identical sequences. Washing is the step in which conditions are set so as to determine a minimum level of similarity between the sequences hybridizing with each other. Generally, from the lowest temperature at which only homologous hybridization occurs, a 1% mismatch between two sequences results in a 1° C. decrease in the melting temperature (Tm) for any chosen SSC concentration. Generally, a doubling of the concentration of SSC results in an increase in the Tₘ of about 17° C. Using these guidelines, the washing temperature can be determined empirically, depending on the level of mismatch sought. Hybridization and wash conditions are explained in Current Protocols in Molecular Biology (Ausubel, F. M. et al., eds., John Wiley & Sons, Inc., 1995, with supplemental updates) on pages 2.10.1 to 2.10.16, and 6.3.1 to 6.3.6.

High stringency conditions can employ hybridization at either (1) 1×SSC (10×SSC=3 M NaCl, 0.3 M Na₃-citrate.2H₂O (88 g/liter), pH to 7.0 with 1 M HCl), 1% SDS (sodium dodecyl sulfate), 0.1-2 mg/ml denatured salmon sperm DNA at 65° C., (2) 1×SSC, 50% formamide, 1% SDS, 0.1-2 mg/ml denatured salmon sperm DNA at 42° C., (3) 1% bovine serum albumen (fraction V), 1 mM Na₂.EDTA, 0.5 M NaHPO₄ (pH 7.2) (1 M NaHPO₄=134 g Na₂HPO₄.7H₂O, 4 ml 85% H₃PO₄ per liter), 7% SDS, 0.1-2 mg/ml denatured salmon sperm DNA at 65° C., (4) 50% formamide, 5×SSC, 0.02 M Tris-HCl (pH 7.6), 1× Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 42° C., (5) 5×SSC, 5× Denhardt's solution, 1% SDS, 100:g/ml denatured salmon sperm DNA at 65° C., or (6) 5×SSC, 5× Denhardt's solution, 50% formamide, 1% SDS, 100:g/ml denatured salmon sperm DNA at 42° C., with high stringency washes of either (1) 0.3-0.1×SSC, 0.1% SDS at 65° C., or (2) 1 mM Na₂EDTA, 40 mM NaHPO₄ (pH 7.2), 1% SDS at 65° C. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2× the number of A and T bases)+(4× the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

Moderate stringency conditions can employ hybridization at either (1) 4×SSC, (10×SSC=3 M NaCl, 0.3 M Na₃-citrate.2H₂O (88 g/liter), pH to 7.0 with 1 M HCl), 1% SDS (sodium dodecyl sulfate), 0.1-2 mg/ml denatured salmon sperm DNA at 65° C., (2) 4×SSC, 50% formamide, 1% SDS, 0.1-2 mg/ml denatured salmon sperm DNA at 42° C., (3) 1% bovine serum albumen (fraction V), 1 mM Na₂.EDTA, 0.5 M NaHPO₄ (pH 7.2) (1 M NaHPO₄=134 g Na₂HPO₄.7H₂O, 4 ml 85% H₃PO₄ per liter), 7% SDS, 0.1-2 mg/ml denatured salmon sperm DNA at 65° C., (4) 50% formamide, 5×SSC, 0.02 M Tris-HCl (pH 7.6), 1× Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 42° C., (5) 5×SSC, 5× Denhardt's solution, 1% SDS, 100 □g/ml denatured salmon sperm DNA at 65° C., or (6) 5×SSC, 5× Denhardt's solution, 50% formamide, 1% SDS, 100:g/ml denatured salmon sperm DNA at 42° C., with moderate stringency washes of 1×SSC, 0.1% SDS at 65° C. The above conditions are intended to be used for DNA-DNA hybrids of 50 base pairs or longer. Where the hybrid is believed to be less than 18 base pairs in length, the hybridization and wash temperatures should be 5-110° C. below that of the calculated Tₘ of the hybrid, where Tₘ in ° C.=(2× the number of A and T bases)+(4× the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

Low stringency conditions can employ hybridization at either (1) 4×SSC, (10×SSC=3 M NaCl, 0.3 M Na₃-citrate.2H₂O (88 g/liter), pH to 7.0 with 1 M HCl), 1% SDS (sodium dodecyl sulfate), 0.1-2 mg/ml denatured salmon sperm DNA at 50° C., (2) 6×SSC, 50% formamide, 1% SDS, 0.1-2 mg/ml denatured salmon sperm DNA at 40° C., (3) 1% bovine serum albumen (fraction V), 1 mM Na₂.EDTA, 0.5 M NaHPO₄ (pH 7.2) (1 M NaHPO₄=134 g Na₂HPO₄.7H₂O, 4 ml 85% H₃PO₄ per liter), 7% SDS, 0.1-2 mg/ml denatured salmon sperm DNA at 50° C., (4) 50% formamide, 5×SSC, 0.02 M Tris-HCl (pH 7.6), 1× Denhardt's solution (100×=10 g Ficoll 400, 10 g polyvinylpyrrolidone, 10 g bovine serum albumin (fraction V), water to 500 ml), 10% dextran sulfate, 1% SDS, 0.1-2 mg/ml denatured salmon sperm DNA at 40° C., (5) 5×SSC, 5× Denhardt's solution, 1% SDS, 100:g/ml denatured salmon sperm DNA at 50° C., or (6) 5×SSC, 5× Denhardt's solution, 50% formamide, 1% SDS, 100:g/ml denatured salmon sperm DNA at 40° C., with low stringency washes of either 2×SSC, 0.1% SDS at 50° C., or (2) 0.5% bovine serum albumin (fraction V), 1 mM Na₂EDTA, 40 mM NaHPO₄ (pH 7.2), 5% SDS. The above conditions are intended to be used for DNA-DNA hybrids of 50 base pairs or longer. Where the hybrid is believed to be less than 18 base pairs in length, the hybridization and wash temperatures should be 5-10° C. below that of the calculated Tₘ of the hybrid, where Tₘ in ° C.=(2× the number of A and T bases)+(4× the number of G and C bases). For hybrids believed to be about 18 to about 49 base pairs in length, the Tₘ in ° C.=(81.5° C.+16.6(log₁₀M)+0.41(% G+C)−0.61 (% formamide)−500/L), where “M” is the molarity of monovalent cations (e.g., Na⁺), and “L” is the length of the hybrid in base pairs.

Detection of MSI-H marker nucleic acids having a sequence that differs from the nucleotide sequences shown in SEQ ID Nos. 1-34, or a sequence complementary thereto, due to degeneracy in the genetic code, are also within the scope of the invention. Such nucleic acids encode functionally equivalent peptides (i.e., a peptide having equivalent or similar biological activity) but differ in sequence from the sequence shown in the sequence listing due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence of a polypeptide. However, it is expected that DNA sequence polymorphisms that do lead to changes in the amino acid sequences of the subject polypeptides will exist among mammals. One skilled in the art will appreciate that these variations in one or more nucleotides (e.g., up to about 3-5% of the nucleotides) of the nucleic acids encoding polypeptides having an activity of a polypeptide may exist among individuals of a given species due to natural allelic variation.

The invention also includes within its scope a polynucleotide probe or primer which hybridizes under stringent conditions (at least about 4×SSC at 65° C., or at least about 4×SSC at 42° C.; see, for example, U.S. Pat. No. 5,707,829, incorporated herein by reference) with at least 15 contiguous nucleotides of one of SEQ ID Nos. 1-34. By this is intended that when at least 15 contiguous nucleotides of SEQ ID Nos. 1-34 is used as a probe, the probe will preferentially hybridize with a gene or mRNA (of the biological material) comprising the complementary sequence, allowing the identification and detection of the nucleic acids of the biological material that uniquely hybridize to the selected probe. Probes of more than 15 nucleotides can be used, but 15 nucleotides represents enough sequence for unique identification.

Constructs of polynucleotides having the sequence of one or more of SEQ ID Nos. 1-34, a portion thereof, or a sequence complementary thereto, and useful, for example for generating a probe, can be produced synthetically, or obtained from natural sources (e.g., human cells) using methods well known to those of skill in the art (see, for example, Sambrook et al., Molecular Cloning: A Laboratory Manual, 2nd Ed. (Cold Spring Harbor Press, Cold Spring Harbor, N.Y. 1989).

In one embodiment, the MSI-H marker polynucleotide sequences of the invention include those sequences which are at least about 70%, 75%, 80%, 90%, 95%, 97%, or 98% identical to one or more of the sequences of SEQ ID NOS 1-34. Alternatively, the invention includes MSI-H marker polypeptides which can be detected and are thus indicative of colorectal cancer in an individual that are encoded by nucleic acid sequences at least about 70%, 75%, 80%, 90%, 95%, 97%, or 98% identical to a mRNA sequence complementary to the nucleic acid sequence of SEQ ID Nos. 1-34. Particularly preferred polypeptides are those of SEQ ID Nos. 35-68, or fragments thereof, or polypeptide sequences which are at least about 70%, 75%, 80%, 90%, 95%, 98% or 99% identical in sequence to the amino acid sequence of one or more of SEQ ID Nos. 35-68.

In addition to a method for detecting colorectal cancer by identifying the overexpression of one or more of the MSI-H marker polynucleotide sequences or MSI-H marker polypeptides in a biological sample from an individual, the invention further comprises a method of detecting cancer by identifying the presence of one or more MSI-H markers in addition to at least one other colorectal cancer associated marker in the same sample (e.g., in the same serum, tissue, or cell sample).

Marker Panels

In one embodiment of the present invention, a biological sample from an individual is screened against a panel of two or more MSI-H markers. That is, a method of detecting MSI-H is provided whereby MSI-H is said to be detected where two or more of the MSI-H marker sequences of SEQ ID Nos 1-34 are identified as overexpressed by at least 2 fold relative to the same sequences in a biological sample known to not be cancerous.

In a further embodiment, one or more of the MSI-H marker sequences of the present invention may be combined in a panel with one or more additional colorectal cancer specific markers, including marker nucleic acid and amino acid sequences. A “colorectal cancer associated marker” useful in the present invention, refers to a polypeptide or nucleic acid sequence which exhibits over- or underexpression of at least 10% in colorectal cancer cells, tissue, or serum obtained from an individual having colorectal cancer, relative to the level of expression in cells, tissue, or serum obtained from an individual that does not have colorectal cancer. Table 2 below shows a list of “colorectal cancer associated markers” useful in the invention (although colorectal cancer associated markers useful in the invention are not limited to those shown in Table 2), and there correspondence with the sequences set forth in the “Sequence listing”.

TABLE 1

| SEQ ID | Gene | | |
|---|---|---|---|
| NO | Symbol | Length | Type |
| 69 | CEACAM5 | 2974 | DNA |
| 70 | AFP | 2032 | DNA |
| 71 | IL8 | 1639 | DNA |
| 72 | SPP1 | 1524 | DNA |
| 73 | KIAA1077 | 5500 | DNA |
| 74 | MMP12 | 1778 | DNA |
| 75 | UBD | 777 | DNA |
| 76 | COL1A1 | 5921 | DNA |
| 77 | LUM | 1804 | DNA |
| 78 | ENC1 | 4827 | DNA |
| 79 | PIGPC1 | 1098 | DNA |
| 80 | GTF3A | 1381 | DNA |
| 81 | CTSB | 1978 | DNA |
| 82 | MCJ | 1074 | DNA |
| 83 | SLC12A2 | 4098 | DNA |
| 84 | C20orf42 | 3120 | DNA |
| 85 | SDBCAG84 | 1337 | DNA |
| 86 | NAP1L1 | 2908 | DNA |
| 87 | OSF-2 | 3213 | DNA |
| 88 | COL6A3 | 10558 | DNA |
| 89 | SPARC | 2133 | DNA |
| 90 | TGFBI | 2691 | DNA |
| 91 | FN1 | 8027 | DNA |
| 92 | COL1A2 | 5084 | DNA |
| 93 | S100A11 | 595 | DNA |
| 94 | LC27 | 2116 | DNA |
| 95 | IRAK1 | 3583 | DNA |
| 96 | IFITM2 | 905 | DNA |
| 97 | TIMP1 | 782 | DNA |
| 98 | IGFBP7 | 1124 | DNA |
| 99 | IFITM1 | 647 | DNA |
| 100 | COL3A1 | 5489 | DNA |
| 101 | IGFBP5 | 1722 | DNA |
| 102 | RegIV | 1200 | DNA |
| 103 | AGR2 | 1701 | DNA |
| 104 | HSPCA | 2259 | DNA |
| 105 | KIAA1199 | 7080 | DNA |
| 106 | MMP1 | 1973 | DNA |
| 107 | MMP7 | 1127 | DNA |
| 108 | TSC | 1163 | DNA |
| 109 | HAIK1 | 2007 | DNA |
| 110 | DAP3 | 1650 | DNA |
| 111 | | 2566 | DNA |
| 112 | | 2067 | DNA |
| 113 | KRT8 | 1752 | DNA |
| 114 | KRT18 | 1412 | DNA |
| 115 | KRT19 | 1407 | DNA |
| 116 | KRT20 | 1723 | DNA |
| 117 | MUC1 | 4139 | DNA |
| 118 | MUC2 | 15720 | DNA |
| 119 | MUC3 | 4707 | DNA |
| 120 | MUC5AC | 4151 | DNA |
| 121 | CGB5 | 880 | DNA |
| 122 | EGFR | 5532 | DNA |
| 123 | ERBB2 | 4530 | DNA |
| 124 | FTH1 | 801 | DNA |
| 125 | FTL | 878 | DNA |
| 126 | ALPP | 2747 | DNA |
| 127 | ODC1 | 2062 | DNA |
| 128 | MUC16 | 3557 | DNA |
| 129 | CEACAM1 | 3464 | DNA |
| 130 | CEACAM3 | 1022 | DNA |
| 131 | CEACAM4 | 1190 | DNA |
| 132 | CEACAM6 | 2249 | DNA |
| 133 | CEACAM7 | 2292 | DNA |
| 134 | CEACAM8 | 2297 | DNA |
| 135 | CA9 | 1552 | DNA |
| 136 | CEACAM5 | 702 | Protein |
| 137 | AFP | 609 | Protein |
| 138 | IL8 | 99 | Protein |
| 139 | SPP1 | 300 | Protein |
| 140 | KIAA1077 | 871 | Protein |
| 141 | MMP12 | 470 | Protein |
| 142 | UBD | 165 | Protein |
| 143 | COL1A1 | 1464 | Protein |
| 144 | LUM | 338 | Protein |
| 145 | ENC1 | 589 | Protein |
| 146 | PIGPC1 | 193 | Protein |
| 147 | GTF3A | 423 | Protein |
| 148 | CTSB | 339 | Protein |
| 149 | MCJ | 150 | Protein |
| 150 | SLC12A2 | 1212 | Protein |
| 151 | C20orf42 | 230 | Protein |
| 152 | SDBCAG84 | 383 | Protein |
| 153 | NAP1L1 | 391 | Protein |
| 154 | OSF-2 | 836 | Protein |
| 155 | COL6A3 | 3176 | Protein |
| 156 | SPARC | 303 | Protein |
| 157 | TGFBI | 683 | Protein |
| 158 | FN1 | 2355 | Protein |
| 159 | COL1A2 | 1366 | Protein |
| 160 | S100A11 | 105 | Protein |
| 161 | LC27 | 283 | Protein |
| 162 | IRAK1 | 712 | Protein |
| 163 | IFITM2 | 132 | Protein |
| 164 | TIMP1 | 207 | Protein |
| 165 | IGFBP7 | 282 | Protein |
| 166 | IFITM1 | 125 | Protein |
| 167 | COL3A1 | 1466 | Protein |
| 168 | IGFBP5 | 272 | Protein |
| 169 | RegIV | 158 | Protein |
| 170 | AGR2 | 175 | Protein |
| 171 | HSPCA | 732 | Protein |
| 172 | KIAA1199 | 1361 | Protein |
| 173 | MMP1 | 469 | Protein |
| 174 | MMP7 | 267 | Protein |
| 175 | TSC | 216 | Protein |
| 176 | HAIK1 | 422 | Protein |
| 177 | DAP3 | 398 | Protein |
| 178 | | 75 | Protein |
| 179 | | 163 | Protein |
| 180 | KRT8 | 483 | Protein |
| 181 | KRT18 | 430 | Protein |
| 182 | KRT19 | 400 | Protein |
| 183 | KRT20 | 424 | Protein |
| 184 | MUC1 | 1255 | Protein |
| 185 | MUC2 | 5179 | Protein |
| 186 | MUC3 | 1217 | Protein |
| 187 | MUC5AC | 1373 | Protein |
| 188 | CGB5 | 165 | Protein |
| 189 | EGFR | 1210 | Protein |
| 190 | ERBB2 | 1255 | Protein |
| 191 | FTH1 | 190 | Protein |
| 192 | FTL | 175 | Protein |
| 193 | ALPP | 535 | Protein |
| 194 | ODC1 | 461 | Protein |
| 195 | MUC16 | 1148 | Protein |
| 196 | CEACAM1 | 526 | Protein |
| 197 | CEACAM3 | 212 | Protein |
| 198 | CEACAM4 | 244 | Protein |
| 199 | CEACAM6 | 344 | Protein |
| 200 | CEACAM7 | 265 | Protein |
| 201 | CEACAM8 | 349 | Protein |
| 202 | CA9 | 459 | Protein |

Additional non-limiting examples of colorectal cancer associated markers, as used in the present invention, may be a carbohydrate epitope present on a polypeptide or nucleic acid molecule and/or an antibody molecule which recognizes and is capable of binding to such an epitope, wherein the carbohydrate epitope is known to be associated with the presence of colorectal cancer in an individual. Such carbohydrate epitopes may be present on more than one unrelated protein or polypeptide. In one embodiment, such a carbohydrate epitope is CA 19-9, also known as sialyl-Lewis^{a}, is a tumor marker defined by a monoclonal antibody as a carbohydrate epitope, related to the blood group antigens, composed of a branching, 5-sugar structure covalently bound to a variety of glycoproteins or glycolipids. The proteins primarily belong to the mucin family and the lipids are usually membrane associated. The CA 19-9 epitope is typically the terminal moiety of a complex, O-linked carbohydrate structure on either macromolecule. Other tumor markers also defined as various carbohydrate epitopes useful in the present invention as a “colorectal cancer associated marker” include CA72-4 which is indicative of the presence of the Tag 72 antigen, which is a triply sialylated Tn antigen on varying protein backbones; Thomsen Freidenreich antigen (TF), which is a sialylated n-acetyl galactosamine moiety O-linked to various peptides; Tn and sialyated Tn (sTn) which is the backbone of the TF antigen without the terminal n-acetyl galactosamine moiety, O-linked to various peptides; CA 50 which is an epitope corresponding to sialylated Lewis A blood group antigen; CA 549 which is a CHO moiety on muc-1; CA 242 which is a sialylated CHO; LASA which is a lipid associated sialic acid, that is, a lipid without a protein associated to it; Du-PAN's 1-5, which are pancreatic associated mucin-like CHO antigens. These useful colon cancer specific antigens and others are known in the art and are described, for example, in “Serological Cancer Markers” Sell, S., Ed. 1992. Humana Press Inc., Totowa, N.J.

Calculation of Sequence Homology

In one embodiment, the present invention relates to the detection of MSI-H in an individual by detecting the presence of a MSI-H marker sequence or a sequence homologous thereto, by using probes and/or primers which are complementary to portions of the MSI-H marker sequence, or are sufficiently homologous to portions of the MSI-H marker sequence to permit hybridization of the probes and/or primers to the MSI-H marker sequence under high stringency conditions. Sequences of the invention are at least 50% homologous to the MSI-H markers of SEQ ID Nos 1-34, and are preferably 60%, 70%, 80%, 90% homologous up to complete sequence identity with MSI-H marker sequences (or optionally to a sequence encoding one or more additional colorectal cancer associated markers).

Sequence identity with respect to any of the sequences presented herein can be determined by a simple “eyeball” comparison (i.e. a strict comparison) of any one or more of the sequences with another sequence to see if that other sequence has, for example, at least 80% sequence identity to the sequence(s).

Relative sequence identity can also be determined by commercially available computer programs that can calculate % identity between two or more sequences using any suitable algorithm for determining identity, using for example default parameters. A typical example of such a computer program is CLUSTAL. Other computer program methods to determine identity and similarity between two sequences include but are not limited to the GCG program package (Devereux et al 1984 Nucleic Acids Research 12: 387) and FASTA (Atschul et al 1990 J Molec Biol 403-410). % homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalizing unduly the overall homology score. This is achieved by inserting “gaps” in the sequence alignment to try to maximize local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affinity gap costs” are typically used that charge a relatively high cost for the existence of a gap and a smaller penalty for each subsequent residue in the gap. This is the most commonly used gap scoring system. High gap penalties will of course produce optimized alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software that can perform sequence comparisons include, but are not limited to, the BLAST package (Ausubel et al., 1995, Short Protocols in Molecular Biology, 3rd Edition, John Wiley & Sons), FASTA (Atschul et al., 1990, J. Mol. Biol., 403-410) and the GENEWORKS suite of comparison tools. Both BLAST and FASTA are available for offline and online searching (Ausubel et al., 1999 supra, pages 7-58 to 7-60).

Although the final % homology can be measured in terms of identity, the alignment process itself is typically not based on an all-or -nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62.

Advantageously, the BLAST algorithm is employed, with parameters set to default values. The BLAST algorithm is described in detail on the World Wide Web at ncbi.nih.gov/BLAST/blast_help.html, which is incorporated herein by reference. The search parameters are defined as follows, and can be advantageously set to the defined default parameters.

Advantageously, “substantial identity” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (Karlin and Altschul 1990, Proc. Natl. Acad. Sci. USA 87:2264-68; Karlin and Altschul, 1993, Proc. Natl. Acad. Sci. USA 90:5873-7; see http://www.ncbi.nih.gov/BLAST/blast_help.html) with a few enhancements. The BLAST programs are tailored for sequence similarity searching, for example to identify homologues to a query sequence. For a discussion of basic issues in similarity searching of sequence databases, see Altschul et al (1994) Nature Genetics 6:119-129.

The five BLAST programs available on the World Wide Web at ncbi.nlm.nih.gov perform the following tasks: blastp—compares an amino acid query sequence against a protein sequence database; blastn—compares a nucleotide query sequence against a nucleotide sequence database; blastx—compares the six-frame conceptual translation products of a nucleotide query sequence (both strands) against a protein sequence database; tblastn—compares a protein query sequence against a nucleotide sequence database dynamically translated in all six reading frames (both strands); tblastx—compares the six-frame translations of a nucleotide query sequence against the six-frame translations of a nucleotide sequence database.

BLAST uses the following search parameters:

HISTOGRAM—Display a histogram of scores for each search; default is yes. (See parameter H in the BLAST Manual).

DESCRIPTIONS—Restricts the number of short descriptions of matching sequences reported to the number specified; default limit is 100 descriptions. (See parameter V in the manual page).

EXPECT—The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF—Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

ALIGNMENTS—Restricts database sequences to the number specified for which high- scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

MATRIX—Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND—Restrict a TBLASTN search to just the top or bottom strand of the database sequences; or restrict a BLASTN, BLASTX or TBLASTX search to just reading frames on the top or bottom strand of the query sequence.

FILTER—Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States (1993) Computers and Chemistry 17:191-201, or, for BLASTN, by the DUST program of Tatusov and Lipman (see http://www.ncbi.nlm.nih.gov). Filtering can eliminate statistically significant but biologically uninteresting reports from the blast output (e.g., hits against common acidic-, basic- or proline- rich regions), leaving the more biologically interesting regions of the query sequence available for specific matching against database sequences.

Low complexity sequence found by a filter program is substituted using the letter “N” in nucleotide sequence (e.g., “NNNNNNNNNNNN”) and the letter “X” in protein sequences (e.g., “XXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi—Causes NCBI gi identifiers to be shown in the output, in addition to the accession and/or locus name.

Most preferably, sequence comparisons are conducted using the simple BLAST search algorithm provided on the World Wide Web at ncbi.nlm.nih.gov/BLAST. In some embodiments of the present invention, no gap penalties are used when determining sequence identity.

Probes and Primers

The nucleotide sequence of MSI-H markers are useful in the present invention for the generation of probes and primers designed for identifying the MSI-H marker nucleic acid sequence in a biological sample such as serum, colon cells or tissue. Nucleotide sequences useful as probes/primers may include all or a portion of SEQ ID Nos. 1-34, or a sequence complementary thereto, or sequences which hybridize under stringent conditions to all or a portion of SEQ ID No. 1-34. For instance, the present invention also provides a probe/primer comprising a substantially purified oligonucleotide, which oligonucleotide comprising a nucleotide sequence that hybridizes under stringent conditions to at least approximately 8, preferably about 12, preferably about 15, preferably about 25, more preferably about 40 consecutive nucleotides up to the full length of the sense or anti-sense sequence of SEQ ID Nos. 1-34, or a sequence complementary thereto, or a naturally occurring mutant thereof. For instance, primers based on the nucleic acid represented in one of SEQ ID Nos 1-34, or a sequence complementary thereto, can be used in a reaction to amplify a template nucleic acid (e.g., the MSI-H marker from which the primer sequence was derived) contained within an mRNA sample derived from a patient clinical sample.

Not only are probes based on the nucleic acid sequence encoding MSI-H marker sequences useful for detecting the MSI-H marker itself, but they can also provide a method for detecting mutations in wild-type MSI-H marker sequences in a patient. Nucleic acid probes which are complementary to a wild-type MSI-H marker sequence and can form mismatches with mutant genes are provided, allowing for detection by enzymatic or chemical cleavage or by shifts in electrophoretic mobility. Likewise, probes based on the subject sequences can be used to detect transcripts or genomic sequences encoding the same or homologous proteins, for use, for example, in prognostic or diagnostic assays. In preferred embodiments, the nucleic acid probe further comprises a label group attached thereto and able to be detected, e.g., the label group is selected from a radioisotope, a fluorescent compound, a chemiluminescent compound, a chromagenic compound, an enzyme, and enzyme co-factor.

Full-length cDNA molecules comprising the disclosed nucleic acids, useful for the generation of probes, primers, or antibodies to the MSI-H marker polypeptide sequences of SEQ ID Nos 35-68 may be obtained as follows. The nucleic acid sequence of MSI-H markers (SEQ ID Nos 1-34) or a portion thereof comprising at least approximately 8, preferably about 12, preferably about 15, preferably about 25, more preferably about 40 nucleotides up to the full length of the sequence of SEQ ID Nos. 1-34, or a sequence complementary thereto, may be used as a hybridization probe to detect hybridizing members of a cDNA library using probe design methods, cloning methods, and clone selection techniques as described in U.S. Pat. No. 5,654,173, “Secreted Proteins and Polynucleotides Encoding Them,” incorporated herein by reference. Libraries of cDNA may be made from selected tissues, such as normal or tumor tissue, or from tissues of a mammal treated with, for example, a pharmaceutical agent. Preferably, the tissue is the same as that used to generate the nucleic acids, as both the nucleic acid and the cDNA represent expressed genes. Alternatively, many cDNA libraries are available commercially. (Sambrook et al., Molecular Cloning: A Laboratory Manual, 2nd Ed. (Cold Spring Harbor Press, Cold Spring Harbor, N.Y. 1989). The choice of cell type for library construction may be made after the identity of the protein encoded by the nucleic acid- related gene is known. This will indicate which tissue and cell types are likely to express the related gene, thereby containing the mRNA for generating the cDNA.

Members of the library that are larger than the nucleic acid, and preferably that contain the whole sequence of the native message, may be obtained. To confirm that the entire cDNA has been obtained, RNA protection experiments may be performed as follows. Hybridization of a full-length cDNA to an mRNA may protect the RNA from RNase degradation. If the cDNA is not full length, then the portions of the mRNA that are not hybridized may be subject to RNase degradation. This may be assayed, as is known in the art, by changes in electrophoretic mobility on polyacrylamide gels, or by detection of released monoribonucleotides. Sambrook et al., Molecular Cloning: A Laboratory Manual, 2nd Ed. (Cold Spring Harbor Press, Cold Spring Harbor, N.Y. 1989). In order to obtain additional sequences 5′ to the end of a partial cDNA, 5′ RACE (PCR Protocols: A Guide to Methods and Applications (Academic Press, Inc. 1990)) may be performed.

Genomic DNA (e.g., MSI-H marker genomic DNA) may be isolated using nucleic acids in a manner similar to the isolation of full-length cDNAs. Briefly, the nucleic acids, or portions thereof, may be used as probes to libraries of genomic DNA. Preferably, the library is obtained from the cell type that was used to generate the nucleic acids. Most preferably, the genomic DNA is obtained from the biological material described herein in the Example. Such libraries may be in vectors suitable for carrying large segments of a genome, such as P1 or YAC, as described in detail in Sambrook et al., pages 9.4-9.30. In addition, genomic sequences can be isolated from human BAC libraries, which are commercially available from Research Genetics, Inc., Huntville, Ala., USA, for example. In order to obtain additional 5′ or 3′ sequences, chromosome walking may be performed, as described in Sambrook et al., such that adjacent and overlapping fragments of genomic DNA are isolated. These may be mapped and pieced together, as is known in the art, using restriction digestion enzymes and DNA ligase.

Using the nucleic acids of the invention, corresponding full length genes can be isolated using both classical and PCR methods to construct and probe cDNA libraries. Using either method, Northern blots, preferably, may be performed on a number of cell types to determine which cell lines express the gene of interest at the highest rate.

Classical methods of constructing cDNA libraries in Sambrook et al., supra. With these methods, cDNA can be produced from mRNA and inserted into viral or expression vectors. Typically, libraries of mRNA comprising poly(A) tails can be produced with poly(T) primers. Similarly, cDNA libraries can be produced using the instant MSI sequences or portions thereof as primers.

PCR methods may be used to amplify the members of a cDNA library that comprise the desired insert. In this case, the desired insert may contain sequence from the full length cDNA that corresponds to the sequence encoding a MSI polypeptide marker of the invention. Such PCR methods include gene trapping and RACE methods.

Gene trapping may entail inserting a member of a cDNA library into a vector. The vector then may be denatured to produce single stranded molecules. Next, a substrate-bound probe, such a biotinylated oligo, may be used to trap cDNA inserts of interest. Biotinylated probes can be linked to an avidin-bound solid substrate. PCR methods can be used to amplify the trapped cDNA. To trap sequences corresponding to the full length genes, the labeled probe sequence may be based on the nucleic acid of SEQ ID Nos. 1-34, or a sequence complementary thereto. Random primers or primers specific to the library vector can be used to amplify the trapped cDNA. Such gene trapping techniques are described in Gruber et al., PCT WO 95/04745 and Gruber et al., U.S. Pat. No. 5,500,356. Kits are commercially available to perform gene trapping experiments from, for example, Life Technologies, Gaithersburg, Md., USA.

“Rapid amplification of cDNA ends,” or RACE, is a PCR method of amplifying cDNAs from a number of different RNAs. The cDNAs may be ligated to an oligonucleotide linker and amplified by PCR using two primers. One primer may be based on sequence from the instant nucleic acids, for which full length sequence is desired, and a second primer may comprise a sequence that hybridizes to the oligonucleotide linker to amplify the cDNA. A description of this method is reported in PCT Pub. No. WO 97/19110.

In preferred embodiments of RACE, a common primer may be designed to anneal to an arbitrary adaptor sequence ligated to cDNA ends (Apte and Siebert, Biotechniques 15:890-893, 1993; Edwards et al., Nuc. Acids Res. 19:5227-5232, 1991). When a single gene-specific RACE primer is paired with the common primer, preferential amplification of sequences between the single gene specific primer and the common primer occurs. Commercial cDNA pools modified for use in RACE are available.

Once the full-length cDNA or gene is obtained, DNA encoding variants can be prepared by site-directed mutagenesis, described in detail in Sambrook 15.3-15.63. The choice of codon or nucleotide to be replaced can be based on the disclosure herein on optional changes in amino acids to achieve altered protein structure and/or function.

As an alternative method to obtaining DNA or RNA from a biological material, such as colon tissue, nucleic acid comprising nucleotides having the sequence of one or more nucleic acids of the invention can be synthesized. Thus, the invention encompasses nucleic acid molecules ranging in length from about 8 nucleotides (corresponding to at least 12 contiguous nucleotides which hybridize under stringent conditions to or are at least 80% identical to the nucleic acid sequence of one of SEQ ID Nos. 1-34, or a sequence complementary thereto) up to a maximum length suitable for one or more biological manipulations, including replication and expression, of the nucleic acid molecule. The invention includes but is not limited to (a) nucleic acid having the size of the full gene represented by each of the MSI-H marker polynucleotide sequences of the invention, or a sequence complementary thereto; (b) the nucleic acid of (a) also comprising at least one additional gene, operably linked to permit expression of a fusion protein; (c) an expression vector comprising (a) or (b); (d) a plasmid comprising (a) or (b); and (e) a recombinant viral particle comprising (a) or (b).

The sequence of a nucleic acid of the present invention is not limited and can be any sequence of A, T, G, and/or C (for DNA) and A, U, G, and/or C (for RNA) or modified bases thereof, including inosine and pseudouridine. The choice of sequence will depend on the desired function and can be dictated by coding regions desired, the intron-like regions desired, and the regulatory regions desired.

Probe Preparation

Prior to hybridization of a probe nucleic acid to a patient sample, the nucleic acid samples must be prepared to facilitate subsequent detection of hybridization. The nucleic acid samples obtained from an individual to be screened for colorectal cancer or MSI are capable of being bound by a nucleic acid probe of complementary sequence through one or more types of chemical bonds, usually through complementary base pairing, usually through hydrogen bond formation.

Probes useful in the invention for hybridizing to and thus identifying the presence of one or more of the MSI-H marker sequences of SEQ ID Nos 1-34, and optionally, at least one additional colorectal cancer associated marker may be designed to hybridize to a polynucleotide molecule derived from an mRNA transcript coding for one or more of the MSI-H marker sequences or optionally, at least one additional colorectal cancer associated marker. As used herein, a “polynucleotide derived from an mRNA transcript” refers to a polynucleotide for which synthesis of the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, suitable target nucleic acid samples include, but are not limited to, mRNA transcripts of a gene or genes (i.e., MSI-H marker or a colorectal cancer associated marker), cDNA reverse transcribed from the mRNA, cRNA transcribed from the cDNA, DNA amplified from a gene or genes, RNA transcribed from amplified DNA, and the like. The polynucleotide probes used herein are preferably designed to hybridize to one or more of the MSI-H marker sequences, or optionally to a sequence encoding at least one other colorectal cancer associated marker.

Nucleic acid probes may be generated using techniques which are well known to those of skill in the art (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987).

In order to measure the hybridization of a probe nucleic acid to a target sequence in a sample, the probe nucleic acid is preferably labeled with a detectable label. Any analytically detectable marker that is attached to or incorporated into a molecule may be used in the invention. An analytically detectable marker refers to any molecule, moiety or atom which is analytically detected and quantified.

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, 35S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The labels may be incorporated into a nucleic acid probe by any of a number of means well known to those of skill in the art. However, in a preferred embodiment, the label is simultaneously incorporated into the probe during an amplification step in the preparation of the probe polynucleotides. Thus, for example, polymerase chain reaction (PCR), or other amplification reaction, with labeled primers or labeled nucleotides will provide a labeled amplification product, and thus a labeled probe.

Alternatively, a label may be added directly to the probe. Means of attaching labels to polynucleotides are well known to those of skill in the art and include, for example nick translation or end-labeling (e.g. with a labeled RNA) and subsequent attachment (ligation) of a polynucleotide linker joining the sample polynucleotide to a label (e.g., a fluorophore).

In a preferred embodiment, the fluorescent modifications are by cyanine dyes e.g. Cy-3/Cy-5 dUTP, Cy-3/Cy-5 dCTP (Amersham Pharmacia) or alexa dyes (Khan, J., Simon, R., Bittner, M., Chen, Y., Leighton, S. B., Pohida, T., Smith, P. D., Jiang, Y., Gooden, G. C., Trent, J. M. & Meltzer, P. S. (1998) Cancer Res. 58, 50095013.).

In a preferred embodiment, a probe nucleic acid which is capable of hybridizing to one or more of the MSI-H marker sequences described herein and a probe nucleic acid which is capable of hybridizing to a nucleic acid sequence encoding at least one additional colorectal cancer associated marker, are co-hybridized to a test sample (e.g., a serum sample). In this embodiment, the two probe samples used for comparison are labeled with different fluorescent dyes which produce distinguishable detection signals, for example, probes hybridizable with a MSI nucleic acid marker sequence are labeled with Cy5 and probes hybridizable with another colorectal cancer associated marker are labeled with Cy3. The differently labeled target samples are hybridized to the same microarray simultaneously.

In a preferred embodiment, a control probe may be co-hybridized to a sample along with a probe for a MSI-H marker sequence and/or a probe for an additional colorectal cancer associated marker, wherein the control probe is capable of hybridizing to a nucleic acid sequence known to be found in the clinical sample, for example, where the clinical sample is a serum sample, a control sequence may be a sequence encoding serum albumin, or fibrinogen.

Antibodies

The invention provides a method for MSI-H detection (and subsequent stratification of patients based on MSI status for prognosis, treatment, and/or monitoring) comprising the step of detecting the presence and overexpression of polypeptide molecules encoded by one or more of the MSI-H marker sequences of SEQ ID Nos 1-34 (and optionally, at least one additional colorectal cancer associated marker) in a biological sample from an individual. In one embodiment, the presence of one or more MSI-H markers , or other marker, in such a sample is detected using a polypeptide ligand which is preferably detectably labeled, and is capable of binding to the MSI-H marker polypeptide, and if present, the other marker, in the sample. In a preferred embodiment, the polypeptide ligand is an antibody. Antibodies of the invention include, but are not limited to, polyclonal, monoclonal, multispecific, human, humanized, or chimeric antibodies, single chain antibodies, Fab fragments, Fv fragments F(ab′) fragments, fragments produced by a Fab expression library, anti-iodiotypic antibodies, or other epitope binding polypeptide. Preferably, an antibody, useful in the present invention for the detection of one or more MSI-H marker polypeptides(and optionally at least one additional colorectal cancer associated marker), is a human antibody or fragment thereof, including scFv, Fab, Fab′, F(ab′), Fd, single chain antibody, of Fv. Antibodies, useful in the invention may include a complete heavy or light chain constant region, or a portion thereof, or an absence thereof. An antibody, useful in the invention, may be obtained from an art recognized host, such as rabbit, mouse, rat, donkey, sheep, goat, guinea pig, camel, horse, or chicken. In one embodiment, an antibody, useful in the invention can be a humanized antibody, in which amino acids have been replaced in the non-antigen binding regions in order to more closely resemble a human antibody, while still retaining the original binding ability. Methods for making humanized antibodies are described in Teng et al., 1983, Proc. Natl. Acad. Sci. USA 80: 7308-7312; Kozbor et al., 1983, Immunology Today 4: 7279; Olsson et al., 1982, Meth. Enzymol. 92: 3-16; WO 92/06193; EP 0239400.

Antibodies of the present invention may be monospecific, dispecific, trispecific, or of greater multispecificity. As such, one or more MSI polypeptide markers and optionally an additional colorectal cancer associated marker useful for the detection of colorectal cancer and/or MSI-H, or optionally, two or more MSI polypeptide markers may be detected with separate antibodies, or may be detected with the same antibody. Alternatively, a multispecific antibody may exhibit different specificities for different epitopes on the same protein. While specificity of an antibody useful in the present invention to either an MSI-H marker or one or more additional colorectal cancer associated markers is preferred, antibodies that bind polypeptides with at least 95%, 90%, 85%, 75%, 65%, 55%, and at least 50% identity to a polypeptide useful in the present invention for the detection of colorectal cancer are also included in the present invention. Also encompassed in the present invention are antibodies which bind to polypeptide molecules which are encoded by one or more nucleic acid sequences which are complementary to, or hybridize to the sequences of SEQ ID Nos. 1-34, or one or more sequences which are complementary to, or hybridize to a nucleic acid sequence which encodes an additional colorectal cancer associated marker as described herein.

Antibodies of the present invention which are useful for the detection of colorectal cancer may further act as agonists or antagonists of the activity of the polypeptide molecules to which they bind, and may thus be useful as therapeutic molecules for the treatment or prevention of colorectal cancer.

An important, but not limiting, role of an antibody of the present invention is to provide for the purification, or detection of one or more MSI polypeptide markers or other colorectal cancer associated markers in a patient sample, including both in vitro and in vivo detection methods. Antibodies useful for the detection of colorectal cancer as described herein do not have to be used alone, and can be fused to other polypeptides, including a heterologous polypeptide at the N- or C-terminus of the antibody polypeptide sequence. For example, an antibody useful in the present invention may be fused with a detectable label to facilitate detection of the antibody when bound to a target polypeptide. Methods for detectably labeling an antibody polypeptide are known to those of skill in the art.

For the production of antibodies useful in the present invention, various hosts including goats, rabbits, rats, mice, etc., may be immunized by injection with the protein products (or any portion, fragment, or oligonucleotide thereof which retains immunogenic properties) of the candidate genes of the invention. Depending on the host species, various adjuvants may be used to increase the immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

Polyclonal antisera or monoclonal antibodies can be made using methods known in the art. A mammal such as a mouse, hamster, or rabbit, can be immunized with an immunogenic form of a MSI-H marker polypeptide, fragment, modified form thereof, or variant form thereof. Alternatively, an animal may be immunized with an immunogenic form of one or more additional colorectal cancer associated marker polypeptides. Techniques for conferring immunogenicity on such molecules include conjugation to carriers or other techniques well known in the art. For example, the immunogenic molecule can be administered in the presence of adjuvant as described above. Immunization can be monitored by detection of antibody titers in plasma or serum. Standard immunoassay procedures can be used with the immunogen as antigen to assess the levels and the specificity of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art (see, e.g., Kohler and Milstein, 1975, Nature 256: 495-497; Kozbor et al., 1983, Immunol. Today 4: 72, Cole et al., 1985, In Monoclonal Antibodies in Cancer Therapy, Allen R. Bliss, Inc., pages 77-96). Additionally, techniques described for the production of single-chain antibodies (U.S. Pat. No. 4,946,778) can be adapted to produce antibodies according to the invention.

Antibody fragments which can specifically bind to a polypeptide of the invention, fragments thereof, modified forms thereof, and variants thereof, also may be generated by known techniques. For example, such fragments include, but are not limited to, F(ab′)₂ fragments which can be produced by pepsin digestion of the antibody molecule and the Fab fragments which can be generated by reducing the disulfide bridges of the F(ab′)₂ fragments. VH regions and FV regions can be expressed in bacteria using phage expression libraries (e.g., Ward et al., 1989, Nature 341: 544-546; Huse et al., 1989, Science 246: 1275-1281; McCafferty et al., 1990, Nature 348: 552-554).

Chimeric antibodies, i.e., antibody molecules that combine a non-human animal variable region and a human constant region also are within the scope of the invention. Chimeric antibody molecules include, for example, the antigen binding domain from an antibody of a mouse, rat, or other species, with human constant regions. Standard methods may be used to make chimeric antibodies containing the immunoglobulin variable region which recognizes the gene product of MSI-H marker antigens of the invention (see, e.g., Morrison et al., 1985, Proc. Natl. Acad. Sci. USA 81: 6851; Takeda et al., 1985, Nature 314: 452; U.S. Pat. No. 4,816,567; U.S. Pat. No. 4,816,397). Vectors and Host Cells

The present invention further provides vectors and plasmids useful for directing the expression of one or more of the MSI-H markers of the invention or other colorectal cancer associated markers, and further provides host cells which express the vectors and plasmids provided herein. Nucleic acid sequences useful for the expression from a vector or plasmid as described below include, but are not limited to any nucleic acid or gene sequence identified as being differentially regulated by the methods described above, and further include therapeutic nucleic acid molecules, such as antisense molecules. The host cell may be any prokaryotic or eukaryotic cell. Ligating the polynucleotide sequence into a gene construct, such as an expression vector, and transforming or transfecting into hosts, either eukaryotic (yeast, avian, insect or mammalian) or prokaryotic (bacterial cells), are standard procedures well known in the art.

Vectors

There is a wide array of vectors known and available in the art that are useful for the expression of differentially expressed nucleic acid molecules according to the invention. The selection of a particular vector clearly depends upon the intended use of the polypeptide encoded by the differentially expressed nucleic acid. For example, the selected vector must be capable of driving expression of the polypeptide in the desired cell type, whether that cell type be prokaryotic or eukaryotic. Many vectors comprise sequences allowing both prokaryotic vector replication and eukaryotic expression of operably linked gene sequences.

Vectors useful according to the invention may be autonomously replicating, that is, the vector, for example, a plasmid, exists extrachromosomally and its replication is not necessarily directly linked to the replication of the host cell's genome. Alternatively, the replication of the vector may be linked to the replication of the host's chromosomal DNA, for example, the vector may be integrated into the chromosome of the host cell as achieved by retroviral vectors.

Vectors useful according to the invention preferably comprise sequences operably linked to the sequence of interest (e.g., an MSI-H marker sequence) that permit the transcription and translation of the sequence. Sequences that permit the transcription of the linked sequence of interest include a promoter and optionally also include an enhancer element or elements permitting the strong expression of the linked sequences. The term “transcriptional regulatory sequences” refers to the combination of a promoter and any additional sequences conferring desired expression characteristics (e.g., high level expression, inducible expression, tissue- or cell-type-specific expression) on an operably linked nucleic acid sequence.

The selected promoter may be any DNA sequence that exhibits transcriptional activity in the selected host cell, and may be derived from a gene normally expressed in the host cell or from a gene normally expressed in other cells or organisms. Examples of promoters include, but are not limited to the following: A) prokaryotic promoters—E. coli lac, tac, or trp promoters, lambda phage PR or PL promoters, bacteriophage T7, T3, Sp6 promoters, B. subtilis alkaline protease promoter, and the B. stearothermophilus maltogenic amylase promoter, etc.; B) eukaryotic promoters - yeast promoters, such as GAL1, GAL4 and other glycolytic gene promoters (see for example, Hitzeman et al., 1980, J. Biol. Chem. 255: 12073-12080; Alber & Kawasaki, 1982, J. Mol. Appl. Gen. 1: 419-434), LEU2 promoter (Martinez-Garcia et al., 1989, Mol Gen Genet. 217: 464-470), alcohol dehydrogenase gene promoters (Young et al., 1982, in Genetic Engineering of Microorganisms for Chemicals, Hollaender et al., eds., Plenum Press, NY), or the TPI1 promoter (U.S. Pat. No. 4,599,311); insect promoters, such as the polyhedrin promoter (U.S. Pat. No. 4,745,051; Vasuvedan et al., 1992, FEBS Lett. 311: 7-11), the P10 promoter (Vlak et al., 1988, J. Gen. Virol. 69: 765-776), the Autographa californica polyhedrosis virus basic protein promoter (EP 397485), the baculovirus immediate-early gene promoter gene 1 promoter (U.S. Pat. Nos. 5,155,037 and 5,162,222), the baculovirus 39K delayed-early gene promoter (also U.S. Pat. Nos. 5,155,037 and 5,162,222) and the OpMNPV immediate early promoter 2; mammalian promoters—the SV40 promoter (Subramani et al., 1981, Mol. Cell. Biol. 1: 854-864), metallothionein promoter (MT-1; Palmiter et al., 1983, Science 222: 809-814), adenovirus 2 major late promoter (Yu et al., 1984, Nucl. Acids Res. 12: 9309-21), cytomegalovirus (CMV) or other viral promoter (Tong et al., 1998, Anticancer Res. 18: 719-725), or even the endogenous promoter of a gene of interest in a particular cell type.

A selected promoter may also be linked to sequences rendering it inducible or tissue- specific. For example, the addition of a tissue-specific enhancer element upstream of a selected promoter may render the promoter more active in a given tissue or cell type. Alternatively, or in addition, inducible expression may be achieved by linking the promoter to any of a number of sequence elements permitting induction by, for example, thermal changes (temperature sensitive), chemical treatment (for example, metal ion- or IPTG-inducible), or the addition of an antibiotic inducing agent (for example, tetracycline).

Regulatable expression is achieved using, for example, expression systems that are drug inducible (e.g., tetracycline, rapamycin or hormone-inducible). Drug-regulatable promoters that are particularly well suited for use in mammalian cells include the tetracycline regulatable promoters, and glucocorticoid steroid-, sex hormone steroid-, ecdysone-, lipopolysaccharide (LPS)- and isopropylthiogalactoside (IPTG)-regulatable promoters. A regulatable expression system for use in mammalian cells should ideally, but not necessarily, involve a transcriptional regulator that binds (or fails to bind) non-mammalian DNA motifs in response to a regulatory agent, and a regulatory sequence that is responsive only to this transcriptional regulator.

Tissue-specific promoters may also be used to advantage in differentially expressed sequence-encoding constructs of the invention. A wide variety of tissue-specific promoters are known. As used herein, the term “tissue-specific” means that a given promoter is transcriptionally active (i.e., directs the expression of linked sequences sufficient to permit detection of the polypeptide product of the promoter) in less than all cells or tissues of an organism. A tissue specific promoter is preferably active in only one cell type, but may, for example, be active in a particular class or lineage of cell types (e.g., hematopoietic cells). A tissue specific promoter useful according to the invention comprises those sequences necessary and sufficient for the expression of an operably linked nucleic acid sequence in a manner or pattern that is essentially the same as the manner or pattern of expression of the gene linked to that promoter in nature. The following is a non-exclusive list of tissue specific promoters and literature references containing the necessary sequences to achieve expression characteristic of those promoters in their respective tissues; the entire content of each of these literature references is incorporated herein by reference. Examples of tissue specific promoters useful in the present invention are as follows:

Bowman et al., 1995 Proc. Natl. Acad. Sci. USA 92, 12115-12119 describe a brain- specific transferrin promoter; the synapsin I promoter is neuron specific (Schoch et al., 1996 J. Biol. Chem. 271, 3317-3323); the nestin promoter is post-mitotic neuron specific (Uetsuki et al., 1996 J. Biol. Chem. 271, 918-924); the neurofilament light promoter is neuron specific (Charron et al., 1995 J. Biol. Chem. 270, 30604-30610); the acetylcholine receptor promoter is neuron specific (Wood et al., 1995 J. Biol. Chem. 270, 30933-30940); and the potassium channel promoter is high-frequency firing neuron specific (Gan et al., 1996 J. Biol. Chem 271, 5859-5865). Any tissue specific transcriptional regulatory sequence known in the art may be used to advantage with a vector encoding a differentially expressed nucleic acid sequence obtained from an animal subjected to pain.

In addition to promoter/enhancer elements, vectors useful according to the invention may further comprise a suitable terminator. Such terminators include, for example, the human growth hormone terminator (Palmiter et al., 1983, supra), or, for yeast or fungal hosts, the TPI1 (Alber & Kawasaki, 1982, supra) or ADH3 terminator (McKnight et al., 1985, EMBO J. 4: 2093-2099).

Vectors useful according to the invention may also comprise polyadenylation sequences (e.g., the SV40 or Ad5E1b poly(A) sequence), and translational enhancer sequences (e.g., those from Adenovirus VA RNAs). Further, a vector useful according to the invention may encode a signal sequence directing the recombinant polypeptide to a particular cellular compartment or, alternatively, may encode a signal directing secretion of the recombinant polypeptide.

a. Plasmid Vectors.

Any plasmid vector that allows expression of a coding sequence of interest (e.g., the coding sequence of a MSI-H marker peptide sequence) in a selected host cell type is acceptable for use according to the invention. A plasmid vector useful in the invention may have any or all of the above-noted characteristics of vectors useful according to the invention. Plasmid vectors useful according to the invention include, but are not limited to the following examples: Bacterial—pQE70, pQE60, pQE-9 (Qiagen) pBs, phagescript, psiX174, pBluescript SK, pBsKS, pNH8a, pNH16a, pNH18a, pNH46a (Stratagene); pTrc99A, pKK223-3, pKK233-3, pDR540, and pRIT5 (Pharmacia); Eukaryotic—pWLneo, pSV2cat, pOG44, pXT1, pSG (Stratagene) pSVK3, pBPV, pMSG, and pSVL (Pharmacia). However, any other plasmid or vector may be used as long as it is replicable and viable in the host.

b. Bacteriophage Vectors.

There are a number of well known bacteriophage-derived vectors useful according to the invention. Foremost among these are the lambda-based vectors, such as Lambda Zap II or Lambda-Zap Express vectors (Stratagene) that allow inducible expression of the polypeptide encoded by the insert. Others include filamentous bacteriophage such as the M13-based family of vectors.

c. Viral Vectors.

A number of different viral vectors are useful according to the invention, and any viral vector that permits the introduction and expression of one or more of the polynucleotides of the invention in cells is acceptable for use in the methods of the invention. Viral vectors that can be used to deliver foreign nucleic acid into cells include but are not limited to retroviral vectors, adenoviral vectors, adeno-associated viral vectors, herpesviral vectors, and Semliki forest viral (alphaviral) vectors. Defective retroviruses are well characterized for use in gene transfer (for a review see Miller, A. D. (1990) Blood 76:271). Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14, and other standard laboratory manuals.

In addition to retroviral vectors, Adenovirus can be manipulated such that it encodes and expresses a gene product of interest but is inactivated in terms of its ability to replicate in a normal lytic viral life cycle (see for example Berkner et al., 1988, BioTechniques 6:616; Rosenfeld et al., 1991, Science 252:431-434; and Rosenfeld et al., 1992, Cell 68:143-155). Suitable adenoviral vectors derived from the adenovirus strain Ad type 5 d1324 or other strains of adenovirus (e.g., Ad2, Ad3, Ad7 etc.) are well known to those skilled in the art. Adeno-associated virus (AAV) is a naturally occurring defective virus that requires another virus, such as an adenovirus or a herpes virus, as a helper virus for efficient replication and a productive life cycle. (For a review see Muzyczka et al., 1992, Curr. Topics in Micro. and Immunol. 158:97-129). An AAV vector such as that described in Traschin et al. (1985, Mol. Cell. Biol. 5:3251-3260) can be used to introduce nucleic acid into cells. A variety of nucleic acids have been introduced into different cell types using AAV vectors (see, for example, Hermonat et al., 1984, Proc. Natl. Acad. Sci. USA 81: 6466-6470; and Traschin et al., 1985, Mol. Cell. Biol. 4: 2072-2081).

Host Cells

Any cell into which a recombinant vector carrying a gene of interest may be introduced and wherein the vector is permitted to drive the expression of the peptide encoded by the differentially expressed sequence is useful according to the invention. Any cell in which a differentially expressed molecule of the invention may be expressed and preferably detected is a suitable host, wherein the host cell is preferably a mammalian cell and more preferably a human cell. Vectors suitable for the introduction of nucleic acid sequences to host cells from a variety of different organisms, both prokaryotic and eukaryotic, are described herein above or known to those skilled in the art.

Host cells may be prokaryotic, such as any of a number of bacterial strains, or may be eukaryotic, such as yeast or other fungal cells, insect or amphibian cells, or mammalian cells including, for example, rodent, simian or human cells. Cells may be primary cultured cells, for example, primary human fibroblasts or keratinocytes, or may be an established cell line, such as NIH3T3, 293T or CHO cells. Further, mammalian cells useful in the present invention may be phenotypically normal or oncogenically transformed. It is assumed that one skilled in the art can readily establish and maintain a chosen host cell type in culture.

Introduction of Vectors to Host Cells

Vectors useful in the present invention may be introduced to selected host cells by any of a number of suitable methods known to those skilled in the art. For example, vector constructs may be introduced to appropriate bacterial cells by infection, in the case of E. coli bacteriophage vector particles such as lambda or M13, or by any of a number of transformation methods for plasmid vectors or for bacteriophage DNA. For example, standard calcium-chloride-mediated bacterial transformation is still commonly used to introduce naked DNA to bacteria (Sambrook et al., 1989, Molecular Cloning, A Laboratory Manual, Cold Spring Harbor Laboratory Press, Cold Spring Harbor, N.Y.), but electroporation may also be used (Ausubel et al., 1988, Current Protocols in Molecular Biology, (John Wiley & Sons, Inc., NY, N.Y.)).

For the introduction of vector constructs to yeast or other fungal cells, chemical transformation methods are generally used (e.g. as described by Rose et al., 1990, Methods in Yeast Genetics, Cold Spring Harbor Laboratory Press, Cold Spring Harbor, N.Y.). For transformation of S. cerevisiae, for example, the cells are treated with lithium acetate to achieve transformation efficiencies of approximately 104 colony-forming units (transformed cells)/μg of DNA. Transformed cells are then isolated on selective media appropriate to the selectable marker used. Alternatively, or in addition, plates or filters lifted from plates may be scanned for GFP fluorescence to identify transformed clones.

For the introduction of vectors comprising a sequence of interest to mammalian cells, the method used will depend upon the form of the vector. Plasmid vectors may be introduced by any of a number of transfection methods, including, for example, lipid-mediated transfection (“lipofection”), DEAE-dextran-mediated transfection, electroporation or calcium phosphate precipitation. These methods are detailed, for example, in Current Protocols in Molecular Biology (Ausubel et al., 1988, John Wiley & Sons, Inc., NY, N.Y.).

Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™ (Stratagene) kits are available. Other companies offering reagents and methods for lipofection include Bio-Rad Laboratories, CLONTECH, Glen Research, InVitrogen, JBL Scientific, MBI Fermentas, PanVera, Promega, Quantum Biotechnologies, Sigma-Aldrich, and Wako Chemicals USA.

Following transfection with a vector of the invention, eukaryotic (e.g., human) cells successfully incorporating the construct (intra- or extrachromosomally) may be selected, as noted above, by either treatment of the transfected population with a selection agent, such as an antibiotic whose resistance gene is encoded by the vector, or by direct screening using, for example, FACS of the cell population or fluorescence scanning of adherent cultures. Frequently, both types of screening may be used, wherein a negative selection is used to enrich for cells taking up the construct and FACS or fluorescence scanning is used to further enrich for cells expressing differentially expressed polynucleotides or to identify specific clones of cells, respectively. For example, a negative selection with the neomycin analog G418 (Life Technologies, Inc.) may be used to identify cells that have received the vector, and fluorescence scanning may be used to identify those cells or clones of cells that express the vector construct to the greatest extent.

Detection Assays

The present invention provides method for detecting colorectal cancer, or alternatively, determining whether a subject is at risk for developing colorectal cancer, or alternatively, determining the presence of MSI-H in a tumor sample, by detecting the overexpression of the disclosed MSI-H markers (and optionally, one or more nucleic acid sequences encoding an additional colorectal cancer associated marker; alternatively, MSI-H polypeptide markers and optionally, at least one additional colorectal cancer associated marker) for the disease or condition encoded thereby.

In clinical applications, human biological samples, can be screened for the presence, absence and/or overexpression of MSI-H markers and/or other colorectal cancer associated markers identified herein. Such samples may comprise tissue samples, whole cells, cell lysates, or isolated nucleic acids, including, for example, needle biopsy cores, surgical resection samples, lymph node tissue, or serum. Methods for obtaining any or all of the above biological samples from an individual are well known to those of skill in the art.

Screening for nucleic acid molecules

In one embodiment, the detection method of the present invention comprises determining whether a biological sample from an individual contains DNA and/or mRNA of a MSI-H marker, and preferably, whether a particular MSI-H marker sequence is overexpressed by at least 2-fold. Techniques for determining the presence of a nucleic acid molecule of interest include, but are not limited to, Northern blot analysis, reverse transcription- polymerase chain reaction (RT-PCR), in situ hybridization, PCR, and quantitative amplification.

Prior to detection of target nucleic acid molecules in a clinical sample, it is preferred to first isolate the nucleic acid (preferably RNA, preferably mRNA) from the sample to facilitate detection of the target sequence. Methods for isolation of RNA from a biological sample are well known in the art, and commercial kits are available for RNA extraction from a biological sample as described herein (e.g., RNeasy kit, QIAGEN, Valencia, Calif.).

Alternatively, total RNA may be extracted from a clinical sample according to the present invention using a commercially available RNA isolation reagent such as Trizol (Invitrogen, Carlsbad, Calif.), following the manufacturers instructions. Purity and integrity of RNA is assessed by absorbance at 260/280 nm and separation of RNA samples on a 1% agarose gel followed by inspection under ultraviolet light.

In one embodiment, following RNA isolation, the RNA may be reverse transcribed to provide a cDNA sample according to methods well known to those of skill in the art (see, e.g., Ausubel et al. (1995), Short Protocols in Molecular Biology, 3^{rd} Ed. John Wiley and Sons, Inc.)

Accordingly, in one aspect, the invention provides probes and primers that specifically hybridize to one or more of the MSI-H marker nucleic acid sequences disclosed herein, or which can hybridize to a nucleic acid molecule encoding an additional colorectal cancer associated marker as described herein. Accordingly, the nucleic acid probes comprise a region of a nucleic acid sequence of SEQ ID Nos 1-34 sufficient to hybridize with a nucleic acid substantially complementary to the sequence of SEQ ID Nos 1-34. Preferred nucleic acid molecules for use as probes/primers can further comprise a region of nucleic acid sequence substantially complementary to the sequence of SEQ ID Nos. 1-34 sufficient to hybridize with the sequence of SEQ ID Nos. 1-34. In addition, nucleic acid sequences useful as probes/primers comprise a nucleotide sequence at least about 8 nucleotides in length, at least about 12 nucleotides in length, preferably at least about 15 nucleotides, more preferably about 25 nucleotides, and most preferably at least 40 nucleotides, and up to all or nearly all of the coding sequence which is complementary to a portion of the coding sequence of a marker nucleic acid sequence, which nucleic acid sequence is represented by SEQ ID Nos: 1-34, or a sequence complementary thereto.

In one embodiment, the method comprises using a nucleic acid probe to determine the presence and overexpression of one or more MSI-H marker nucleic acid molecules in a clinical sample (such as a serum sample or a nucleic acid sample extracted therefrom). Specifically, the method comprises:
1. Providing a nucleic acid probe comprising a nucleotide sequence at least about 8 nucleotides in length, at least about 12 nucleotides in length, preferably at least about 15 nucleotides, more preferably about 25 nucleotides, and most preferably at least about 40 nucleotides, and up to all or nearly all of the coding sequence which is complementary to a portion of the coding sequence of a nucleic acid sequence represented by SEQ ID Nos: 1-34, or a sequence complementary thereto;
2. Obtaining a biological sample from a patient potentially comprising a MSI-H marker nucleic acid sequence;
3. Providing a second clinical sample from an individual known to not have colorectal cancer and/or MSI;
4. Contacting the nucleic acid probe under stringent conditions with RNA of each of said first and second biological samples (e.g., in a Northern blot or in situ hybridization assay); and
5. Comparing (a) the amount of hybridization of the probe with RNA of the first biological sample, with (b) the amount of hybridization of the probe with RNA of the second biological sample; wherein a statistically significant difference in the amount of hybridization with the RNA of the first clinical sample as compared to the amount of hybridization with the RNA of the second biological sample is indicative of the presence of a MSI-H marker sequence in the first biological sample.

Alternatively, the present invention provides a method for detection of MSI-H markers comprising in situ hybridization detection of MSI-H marker nucleic acid sequences with a probe derived from a nucleic acid sequence represented by SEQ ID Nos: 1-34, or a sequence complementary thereto. Preferably, the hybridization probe is detectably labeled. The method comprises contacting the labeled hybridization probe with a tissue or cell sample from an individual suspected of having colorectal cancer, washing off any unbound probe, and detecting the signal produced by the detectable label, wherein the detection of the detectable signal is indicative of the presence of an MSI-H marker sequence in the sample, and thus permits the detection of colorectal cancer and/or MSI. Alternatively, the tissue or cell is additionally hybridized with a detectably labeled nucleic acid probe which is capable of specifically hybridizing with a nucleic acid sequence that encodes at least one additional colorectal cancer associated marker. Detection of the second detectably labeled probe is thus indicative of the presence of the additional colorectal cancer associated marker in the sample, and in conjunction with the detection of MSI-H marker sequence, permits the detection of colorectal cancer and/or MSI in the individual. Specific methods for in situ hybridization are well known in the art.

Alternatively, methods such as PCR, Northern analysis, and Taqman may be used to detect and/or quantitate the expression (and overexpression) of a MSI-H marker nucleic acid sequence in a biological sample. In one embodiment, reverse transcription PCR (RT-PCR) is performed using primers designed to specifically hybridize to a predetermined portion of the MSI-H marker sequence isolated from a biological sample. Generation of a PCR product by such a reaction is thus indicative of the presence of the MSI-H marker sequence in the sample. The technique of designing primers for PCR amplification is well known in the art. Oligonucleotide primers and probes are 5 to 100 nucleotides in length, ideally from 17 to 40 nucleotides, although primers and probes of different length are of use. Primers for amplification are preferably about 17-25 nucleotides. Primers useful according to the invention are also designed to have a particular melting temperature (Tm) by the method of melting temperature estimation. Commercial programs, including Oligo™ (MBI, Cascade, CO), Primer Design and programs available on the internet, including Primer3 and Oligo Calculator can be used to calculate a Tₘ of a nucleic acid sequence useful according to the invention. Preferably, the Tₘ of an amplification primer useful according to the invention, as calculated for example by Oligo Calculator, is preferably between about 45 and 65° C. and more preferably between about 50 and 60° C. Preferably, the Tₘ of a probe useful according to the invention is 7° C. higher than the Tₘ of the corresponding amplification primers. It is preferred that, following generation of cDNA by RT-PCR, the cDNA fragment is cloned into an appropriate sequencing vector, such as a PCRII vector (TA cloning kit; Invitrogen). The identity of each cloned fragment is then confirmed by sequencing in both directions. It is expected that the sequence obtained from sequencing would be the same as the known sequence of the MSI-H marker sequences as described herein.

Alternatively, the presence of MSI-H marker mRNA sequence may be detected by Northern analysis. Sequence confirmed cDNAs, that is, cDNAs representing the MSI-H marker sequences of the invention (or alternatively an additional colorectal cancer associated marker) are used to produce ³²P-labeled cDNA probes using techniques well known in the art (see, for example, Ausubel, supra). Labeled probes for Northern analysis may also be produced using commercially available kits (Prime-It Kit, Stratagene, La Jolla, Calif.). Northern analysis of total RNA obtained from a clinical sample may be performed using classically described techniques. For example, total RNA samples are denatured with formaldehyde/formamide and run for two hours in a 1% agarose, MOPS-acetate-EDTA gel. RNA is then transferred to nitrocellulose membrane by upward capillary action and fixed by UV cross-linkage. Membranes are pre-hybridized for at least 90 minutes and hybridized overnight at 42° C. Post hybridization washes are performed as known in the art (Ausubel, supra). The membrane is then exposed to x-ray film overnight with an intensifying screen at -80° C. Labeled membranes are then visualized after exposure to film. The signal produced on the x-ray film by the radiolabeled cDNA probes can then be quantified using any technique known in the art, such as scanning the film and quantifying the relative pixel intensity using a computer program such as NIH Image (National Institutes of Health, Bethesda, Md.), wherein the detection of hybridization of a MSI-H marker-specific probe to the clinical sample is indicative of the presence of the MSI-H marker and thus may be used to detect colorectal cancer.

In an alternate embodiment, the presence and optionally the quantity of MSI-H markers in a clinical sample may be determined using the Taqman™ (Perkin-Elmer, Foster City, Calif.) technique, which is performed with a transcript-specific antisense probe (i.e., a probe capable of specifically hybridizing to a MSI-H marker sequence as described herein). This probe is specific for a MSI-H marker PCR product and is prepared with a quencher and fluorescent reporter probe complexed to the 5′ end of the oligonucleotide. Different fluorescent markers can be attached to different reporters, allowing for measurement of two products in one reaction (e.g., measurement of two or more MSI-H marker sequences; or alternatively, one MSI-H marker sequence and at least one additional colorectal cancer associated marker). When Taq DNA polymerase is activated, it cleaves off the fluorescent reporters by its 5′-to-3′ nucleolytic activity. The reporters, now free of the quenchers, fluoresce. The color change is proportional to the amount of each specific product and is measured by fluorometer; therefore, the amount of each color can be measured and the RT- PCR product can be quantified. The PCR reactions can be performed in 96 well plates so that samples derived from many individuals can be processed and measured simultaneously. The Taqman™ system has the additional advantage of not requiring gel electrophoresis and allows for quantification when used with a standard curve. The Taqman method thus permits the quantification of the expression of MSI-H marker sequences of the invention, and permits a determination of overexpression of such sequences.

Screening for Polypeptide Molecules

The MSI-H marker-specific and colorectal cancer marker-specific antibodies described above may be used to detect the presence of MSI-H marker polypeptides encoded by SEQ ID Nos 1-34 or an additional colorectal cancer associated marker in a biological sample by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4 C, adding protein A and/or protein G sepharose beads to the cell lysate, incubating for about an hour or more at 4 C, washing the beads in lysis buffer and resuspending the beads in SDS/sample buffer. In the case of immunonprecipitation of a serum sample, however the above protocol is carried out absent the cell lysis step. The ability of the antibody to immunoprecipitate an MSI-H marker polypeptide (or other colorectal cancer marker) antigen can be assessed by, e.g., western blot analysis. The parameters that can be modified to increase the binding of the antibody to an antigen and decrease the background (e.g., preclearing the cell lysate with sepharose beads) are well known to those of skill in the art (Ausubel et al, supra).

MSI-H marker polypeptides, and optionally one or more additional colorectal cancer associated markers may be detected in a patient biological sample using Western blot analysis. Briefly, Western blot analysis comprises preparing protein samples, electrophoresis of the protein samples in a polyacrylamide gel (e.g., 8%-20% SDS-PAGE), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an antihuman antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. Methods for the optimization of such an analysis are well known in the art (Ausubel, et al., supra).

Alternatively, the presence of MSI-H marker polypeptides and optionally one or more additional colorectal cancer associated markers in a clinical sample may be detected by ELISA. ELISAs comprise preparing antigen, coating the well of a 96 well microtiter plate (or other suitable container) with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest, that is, the antibody which will bind to the MSI-H marker peptide or a second colorectal cancer associated marker) conjugated to a detectable compound may be added to the well. Further, instead of coating the well with the antigen, the antibody may be coated to the well. In this case, a second antibody conjugated to a detectable compound may be added following the addition of the antigen of interest to the coated well. This method may be modified or optimized according techniques which are known to those of skill in the art.

The binding affinity of an antibody to an antigen and the off-rate of an antibody/antigen interaction can be determined by competitive binding assays. One example of such an assay is a radioimmunoassay comprising the incubation of labeled antigen (e.g., an MSI-H marker sequence labeled with 3H or 125I) with an anti-MSI-H marker antibody in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., 3H or 125I) in the presence of increasing amounts of an unlabeled second antibody.

Preferably, the above detection assays re be carried out using antibodies to detect the protein product encoded by a nucleic acid having the sequence of SEQ ID Nos: 1-34, or a sequence complementary thereto. Preferably, the protein product has the sequence of one or more of SEQ ID Nos. 35-68. In addition, the above detection assays may be conducted using one or more antibodies which specifically recognize and bind to at least one additional colorectal cancer associated marker. Accordingly, in one embodiment, the assay would include contacting the proteins of the test cell with an antibody specific for the gene product of a nucleic acid represented by SEQ ID Nos: 1-34, or a sequence complementary thereto, and determining the approximate amount of immunocomplex formation by the antibody and the proteins of the test cell, wherein a detection of such an immunocomplex is indicative of the presence of the antigen, and thus, permits the detection of colorectal cancer.

Immunoassays, useful in the present invention include those described above, and can also include both homogeneous and heterogeneous procedures such as fluorescence polarization immunoassay (FPIA), fluorescence immunoassay (FIA), enzyme immunoassay (EIA), and nephelometric inhibition immunoassay (NIA).

In another embodiment, the level of the encoded product, i.e., the product encoded by SEQ ID Nos 1-34, or a sequence complementary thereto, in a biological fluid (e.g., blood or urine) of a patient may be determined as a way of monitoring the level of expression of the marker nucleic acid sequence in cells of that patient. Such a method would include the steps of obtaining a sample of a biological fluid from the patient, contacting the sample (or proteins from the sample) with an antibody specific for a encoded marker polypeptide, and determining the amount of immune complex formation by the antibody, with the amount of immune complex formation being indicative of the level of the marker encoded product in the sample. This determination is particularly instructive when compared to the amount of immune complex formation by the same antibody in a control sample taken from a normal individual or in one or more samples previously or subsequently obtained from the same person.

In another embodiment, the method can be used to determine the amount of marker polypeptide present in a cell, which in turn can be correlated with progression of a hyperproliferative disorder, e.g., colorectal cancer. The level of the marker polypeptide can be used predictively to evaluate whether a sample of cells contains cells which are, or are predisposed towards becoming, transformed cells. Moreover, the subject method can be used to assess the phenotype of cells which are known to be transformed, the phenotyping results being useful in planning a particular therapeutic regimen. For instance, very high levels of the marker polypeptide in sample cells is a powerful diagnostic and prognostic marker for a cancer, such as colorectal cancer. The observation of marker polypeptide level can be utilized in decisions regarding, e.g., the use of more aggressive therapies.

As set out above, one aspect of the present invention relates to detection assays for determining, in the context of cells isolated from a patient, if the level of a marker polypeptide is significantly reduced in the sample cells. The term “significantly reduced” refers to a cell phenotype wherein the cell possesses a reduced cellular amount of the marker polypeptide relative to a normal cell of similar tissue origin. For example, a cell may have less than about 50%, 25%, 10%, or 5% of the marker polypeptide that a normal control cell. In particular, the assay evaluates the level of marker polypeptide in the test cells, and, preferably, compares the measured level with marker polypeptide detected in at least one control cell, e.g., a normal cell and/or a transformed cell of known phenotype.

Of particular importance to the subject invention is the ability to quantitate the level of normal or abnormal MSI-H marker expression. The expression of MSI-H markers, and/or the level of expression of MSI-H markers can be used predictively to evaluate whether a patient is predisposed towards developing colorectal cancer, or for determining the severity of colorectal cancer, or for determining the presence of tumors having microsatellite instability.

In one embodiment, tissue samples may be used to measure MSI-H marker peptide expression by immunohistochemical staining which may be used to determine the number of cells (i.e., colon cells) expressing a given MSI-H marker polypeptide. For such staining, a multiblock of tissue is taken from the biopsy or other tissue sample and subjected to proteolytic hydrolysis, employing such agents as protease K or pepsin. In certain embodiments, it may be desirable to isolate a nuclear fraction from the sample cells and detect the level of the marker polypeptide in the nuclear fraction.

The tissue samples are fixed by treatment with a reagent such as formalin, glutaraldehyde, methanol, or the like. The samples are then incubated with an antibody, preferably a monoclonal antibody, with binding specificity for a given MSI-H marker polypeptide (or a plurality of MSI-H marker polypeptides) and optionally an additional colorectal cancer associated marker. This antibody may be conjugated to a label for subsequent detection of binding. Samples are incubated for a time sufficient for formation of the immunocomplexes. Binding of the antibody is then detected by virtue of a label conjugated to this antibody. Where the antibody is unlabeled, a second labeled antibody may be employed, e.g., which is specific for the isotype of the anti-marker polypeptide antibody. Examples of labels which may be employed include radionuclides, fluorophores, chemiluminescent labels, enzymes and the like.

Where enzymes are employed, the substrate for the enzyme may be added to the samples to provide a colored or fluorescent product. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. Other assays, known to those of skill in the art for determining the presence and/or quantity of a polypeptide in a sample (either serum or tissue) are also encompassed by the present invention.

Prognosis, Staging, and Monitoring of Cancer

In one aspect, the present invention provides methods for determining cancer prognosis and stage based on examining the expression levels of the MSI-H marker sequences and polypeptides using the methods described in the present invention. If cancer is detected in a subject using a technique other than by determining the expression levels of the marker sequences, then the differential expression level of the MSI-H marker sequences can be used to determine the prognosis and stage for the subject. As used herein, prognosis refers to the prediction of the probable course and outcome of a disease.

It is generally accepted in the art that the MSI status of a colorectal cancer tumor can be predictive of clinical outcome and survival, as well as a patient's response to a particular therapy. For example, patients with MSI-H colon tumors have been shown to have increased survival rates compared with patients having MSS or MSI-L colorectal cancer tumors following 5-flurouracil/levamisole and fluoropyrimidine-based chemotherapy (Elaleh et al., 2000 Oncology 58:52; Elsaley and Iacopetta, 2001, Clinical Colorectal Cancer, 1:104), and that, overall, MSI-H status of colorectal tumors may be indicative of greater sensitivity of such tumors to chemical intervention (See, e.g., Liang et al., 2002, Int. J. Cancer 101:519). In addition, MSI-H colorectal tumors are associated with improved overall survival relative to MSS or MSI-L tumors (Gryfe et al, 2000, New England J. Med. 342:69; Ward et al., 2001, Gut 48:821), and are associated with earlier stage colorectal cancer (Kim et al., 1994, Am. J. Pathol. 145:148; Gafa et al., 2000, Cancer 89:2025; Jemvall et al., 1999, Eur. J. Cancer 35:197). Accordingly, the MSI-H status of colorectal tumors, as determined using the overexpression of the MSI-H marker sequences described herein, may be used as a prognostic indicator, may be used to design or select treatment regimes for a given patient, and may be used to stage colorectal cancers.

In general, methods used for prognosis or stage of cancer involve comparison of the amount of the MSI-H marker sequences in a sample of interest with that of a control to detect relative differences in the expression of the MSI-H marker sequences, wherein the difference can be measured qualitatively and/or quantitatively. For example, the expression levels of one or more MSI-H marker RNAs or polypeptides can be compared with the expression levels of the same MSI-H marker RNAs or polypeptides in cancer free or normal samples. Alternatively, the expression levels of one or more MSI-H marker RNAs or polypeptides can also be compared with the expression levels of the same MSI-H marker RNAs or polypeptides observed in cancers that are known not to progress. In addition, the expression levels of one or more MSI-H marker RNAs or polypeptides can also be compared with the expression levels of the same MSI-H marker RNAs or polypeptides observed in cancers that are known to progress and/or metastasize.

Also, as used herein, cancer stage refers to the sequence of the events, in which cancer develops and causes symptoms. In addition, staging is a process used to describe how advanced the cancerous state is in patient. Staging systems vary with the types of cancer, but generally involve the following “TNM” system: the type of tumor, indicated by T; whether the cancer has metastasized to nearby lymph nodes, indicated by N; and whether the cancer has metastasized to more distant parts of the body, indicated by M. Generally, if a cancer is only detectable in the area of the primary lesion without having spread to any lymph nodes it is called Stage I. If it has spread only to the closest lymph nodes, it is called Stage II. In Stage III, the cancer has generally spread to the lymph nodes in near proximity to the site of the primary lesion. Cancers that have spread to a distant part of the body, such as the liver, bone, brain or other site, are Stage IV, the most advanced stage. Methods of the present invention are useful in assaying the staging of cancer. The staging of cancer can be accomplished by determining the expression levels of one or more MSI-H marker nucleic acid or polypeptide relative to a reference expression levels of the same MSI-H marker nucleic acid or polypeptide. The reference expression levels of the marker RNAs or polypeptides can be that from cancer free or healthy or cancer samples, wherein the cancer can be at different stages in development, or alternatively, may be derived from an adjacent portion of the colon which is not infiltrated by tumor.

The present invention further provides methods of monitoring cancer progression or recurrence by measuring the expression levels of the MSI-H marker nucleic acids or polypeptides over the time. In one embodiment, the methods comprise:

(1). detecting in a biological sample of the subject at a first point in time, the expression of one or more nucleic acid sequences comprising one or more nucleic acid sequences selected from the group consisting of SEQ ID NOs: 1-34;

(2). repeating step (a) at a subsequent point in time; and

(3). comparing the expression level detected in steps (a) and (b), wherein a change in the expression level is indicative of progression of cancer or a pre-malignant condition thereof in the subject.

In another embodiment, the methods comprise:

(1). detecting in a biological sample of the subject at a first point in time, the expression of one or more polypeptides comprising one or more polypeptide sequences selected from the group consisting of SEQ ID NOs: 35-68;

(2). repeating step (a) at a subsequent point in time; and

(3). comparing the expression level detected in steps (a) and (b), wherein a change in the expression level is indicative of progression of cancer or a pre-malignant condition thereof in the subject.

For example, elevated expression levels of one or more over-expressed MSI-H marker nucleotides or polypeptides, or reduced expression levels of one or more under-expressed MSI-H marker nucleotides or polypeptides in a subsequent point in time relative to an earlier point in time, indicate that the cancer is progressing to a more severe stage. On the other hand, reduced expression levels of one or more over-expressed MSI-H marker nucleotides or polypeptides, or elevated expression levels of one or more under-expressed MSI-H marker nucleotides or polypeptides in a subsequent point in time relative to an earlier point in time, indicate that the cancer is not progressing or is progressing slowly.

The methods used in prognosis, staging, and monitoring cancer can be applied to various types of cancer. Examples of cancer include but not limited to, adenocarcinoma, lymphoma, blastoma, melanoma, sarcoma, and leukemia. More particularly, examples of cancer also include squamous cell cancer, small-cell lung cancer, non-small cell lung cancer, gastrointestinal cancer, Hodgkin's and non-Hodgkin's lymphoma, pancreatic cancer, glioblastoma, cervical cancer, ovarian cancer, liver cancer such as hepatic carcinoma and hepatoma, bladder cancer, breast cancer, colon cancer, colorectal cancer, endometrial carcinoma, salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer. Preferably, the cancers include breast, colon, and lung cancer. More preferably, the cancer is colon cancer, and the MSI-H marker sequences are the ones comprising a nucleic acid sequence selected from the group consisting of SEQ ID NOs: 1-34.

Efficacy of Therapy and Therapeutic Compositions

Because MSI status permits one of skill in the art to make determinations regarding treatment strategies, in one aspect, the present invention also provides methods that permit the assessment and/or monitoring of patients who will be likely to benefit from both traditional and non-traditional treatments and therapies for cancers, particularly colon cancer. The present invention thus embraces testing, screening and monitoring of patients undergoing anti-cancer treatments and therapies, used alone, in combination with each other, and/or in combination with anti-cancer drugs, anti-neoplastic agents, chemotherapeutics and/or radiation and/or surgery, to treat cancer patients.

An advantage of the present invention is the ability to monitor, or screen over time, those patients who can benefit from one, or several, of the available cancer therapies, and preferably, to monitor patients receiving a particular type of therapy, or a combination therapy, over time to determine how the patient is faring from the treatment(s), if a change, alteration, or cessation of treatment is warranted; if the patient's disease has been reduced, ameliorated, or lessened; or if the patient's disease state or stage has progressed, or become metastatic or invasive. The cancer treatments embraced herein also include surgeries to remove or reduce in size a tumor, or tumor burden, in a patient. Accordingly, the methods of the invention are useful to monitor patient progress and disease status post-surgery.

The identification of the correct patients for a cancer therapy according to this invention can provide an increase in the efficacy of the treatment and can avoid subjecting a patient to unwanted and life-threatening side effects of the therapy. By the same token, the ability to monitor a patient undergoing a course of therapy using the methods of the present invention can determine whether a patient is adequately responding to therapy over time, to determine if dosage or amount or mode of delivery should be altered or adjusted, and to ascertain if a patient is improving during therapy, or is regressing or is entering a more severe or advanced stage of disease, including invasion or metastasis, as discussed further herein.

A method of monitoring according to this invention reflects the serial, or sequential, testing or analysis of a cancer patient by testing or analyzing the patient's body fluid sample over a period of time, such as during the course of treatment or therapy, or during the course of the patient's disease. For instance, in serial testing, the same patient provides a body fluid sample, e.g., serum or plasma, or has sample taken, for the purpose of observing, checking, or examining the expression levels of one or more of the MSI-H markers (DNA, RNA or polypeptide) of the invention in the patient by measuring the levels of one or more of these MSI-H markers during the course of treatment, and/or during the course of the disease, according to the methods of the invention.

Similarly, a patient can be screened over time to assess the levels of one or more of the MSI-H markers in a biological sample for the purposes of determining the status of his or her disease and/or the efficacy, reaction, and response to cancer or neoplastic disease treatments or therapies that he or she is undergoing. It will be appreciated that one or more pretreatment sample(s) is/are optimally taken from a patient prior to a course of treatment or therapy, or at the start of the treatment or therapy, to assist in the analysis and evaluation of patient progress and/or response at one or more later points in time during the period that the patient is receiving treatment and undergoing clinical and medical evaluation.

In monitoring a patient's levels of one or more of the MSI-H markers of the invention over a period of time, which may be days, weeks, months, and in some cases, years, or various intervals thereof, the patient's body fluid sample, e.g., a serum or plasma sample, is collected at intervals, as determined by the practitioner, such as a physician or clinician, to determine the levels of one or more of the MSI-H markers in the cancer patient compared to the respective levels of one or more of these analytes in normal individuals over the course or treatment or disease. For example, patient samples can be taken and monitored every month, every two months, or combinations of one, two, or three month intervals according to the invention. Quarterly, or more frequent monitoring of patient samples, is advisable.

The levels of the one or more MSI-H markers found in the patient are compared with the respective levels of the one or more of these markers in normal individuals, and with the patient's own MSI-H marker levels, for example, obtained from prior testing periods, to determine treatment or disease progress or outcome. Accordingly, use of the patient's own MSI-H marker levels monitored over time can provide, for comparison purposes, the patient's own values as an internal personal control for long-term monitoring of marker levels, and thus cancer presence and/or progression. As described herein, following a course of treatment or disease, the determination of an increase or a decrease in one or more of the MSI-H marker levels in the cancer patient over time compared to the respective levels of one or more of these MSI-H markers in normal individuals reflects the ability to determine the severity or stage of a patient's cancer, or the progress, or lack thereof, in the course or outcome of a patient's cancer therapy or treatment.

Increases or decreases in the levels of the MSI-H markers in cancer patients are determined by comparing the values obtained from analyzing cancer patient samples compared to the normal control range expression levels. A MSI-H marker is said to be over- expressed if expression of the marker is at least 2 fold greater in the cancer patient relative to a normal control, and a MSI-H marker is said to be under expressed if the expression of the marker is at least 2 fold greater in the normal control relative to in the cancer patient.

In monitoring a patient over time, a reduction in the levels of one or more of a patient's MSI-H marker levels from increased levels (i.e., at least 2 fold over-expressed) compared to normal range values to levels at or near to the levels of the analytes found in normal individuals is indicative of treatment progress or efficacy, and/or disease improvement, remission, tumor reduction or elimination, and the like. Likewise, in all of the methods described in the embodiments of this invention, a determination of a reduction of one or more of a patient's MSI-H marker levels from an elevated level (i.e., at least 2 fold over- expressed) to, or approximately to, the respective levels of one or more of these analytes found in normal individuals provides a further aspect of the methods of the invention, in which a patient's improvement, recovery or remission, and/or treatment progress or efficacy, is able to be ascertained over time following performance of the method.

Another embodiment of the present invention encompasses a method of monitoring a cancer patient's course of disease, or the efficacy of a cancer patient's treatment or therapy. The patient's treatment or therapy can involve traditional therapies, such as hormone therapy, chemotherapeutic drug therapy, radiation, or novel therapies, or a combination of any of the foregoing. The method involves measuring levels of one or more MSI-H markers in a body fluid sample of the cancer patient and determining if the levels of one or more of the markers in the patient's sample are changed by at least 2 fold compared to the respective levels of one or more of these analytes in normal controls during the course of disease or cancer treatment. In accordance with the method, a change in the levels of the MSI-H marker in the cancer patient compared to the respective levels of the MSI-H marker in normal controls is indicative of a change in stage, grade, severity or progression of the patient's cancer and/or a lack of efficacy or benefit of the cancer treatment or therapy provided to the patient during a course of treatment, e.g., poor treatment or clinical outcome.

As will be understood by the skilled practitioner in the art, the monitoring method according to this invention is preferably, performed in a serial or sequential fashion, using samples taken from a patient during the course of disease, or a disease treatment regimen, (e.g., after a number of days, weeks, months, or occasionally, years, or various multiples of these intervals) to allow a determination of disease progression or outcome, and/or treatment efficacy or outcome. If the sample is amenable to freezing or cold storage, the samples may be taken from a patient (or normal individual) and stored for a period of time prior to analysis.

In another of its embodiments, the present invention encompasses the determination of the amounts or levels of one or more additional cancer markers in conjunction with the determination of the levels of one or more of the markers of the invention in a sample to be analyzed.

The present invention also includes a method of assessing the efficacy of a test composition for inhibiting cancers, such as colon cancer. As described above, differential expression levels of the MSI-H marker sequences of the invention correlate with the cancerous state of cancer cells, particularly colon cancer cells. It is recognized that changes in the expression levels of the MSI-H marker sequences of the present invention may result from, or induce the cancerous state of cells. Thus, composition which inhibit cancer in a patient will cause the expression levels of the MSI-H marker sequences to change to a level near the normal level of expression for the marker sequences. The method thus comprises comparing expression levels of one or more MSI-H marker sequences in a first biological sample maintained in the presence of a test composition with those of the same MSI-H marker sequences in a second biological sample maintained in the absence of the test composition. A significant difference in the expression levels of one or more marker sequences is an indication that the test composition inhibits the cancer. In a preferred embodiment, the cancer is colon cancer, and the MSI-H marker sequences are the ones listed in SEQ ID Nos 1-34 and/or 35-68. In another embodiment, the cell samples may be aliquots of a single sample obtained from either a healthy subject or a patient with cancerous conditions.

Modulators of the Marker Sequences

It is recognized that changes in the expression levels of the MSI-H marker sequences likely induce, maintain, and promote the cancerous state of cells. Thus, another aspect of the present invention is directed to the modulators of the MSI-H marker sequences capable of modulating the differentiation and proliferation of cells. In this regard, the present invention provides assays for determining compounds that modulate the expression of the marker sequences. The compounds can be used to modulate the biological activity of the polypeptides encoded by the MSI-H marker sequences or the MSI-H marker sequences themselves. Compounds can also be useful in a variety of different environments, including as medicinal agents to treat or prevent disorders associated with cancer.

Methods of identifying compounds generally comprise steps in which a compound is placed in contact with a MSI-H marker sequence, its transcription product, its translation product, or other target, and determination of whether the compound modulates the marker sequence. For modulating the expression of a MSI-H marker sequence, a method can comprise, in any effective order, one or more of the following steps, e.g., contacting the marker sequence (e.g., in a cell population) with a test compound under conditions effective for said test compound to modulate the expression of the marker sequence, and determining whether said test agent modulates said sequence. A compound can modulate expression of a sequence at any level, including transcription (e.g., by modulating the promoter), translation, and/or maintenance of the nucleic acid (e.g., degradation, stability, etc.) in the cell.

For modulating the biological activity of polypeptides, a method can comprise, in any effective order, one or more of the following steps, e.g., contacting a polypeptide (e.g., in a cell, lysate, or isolated) with a test compound under conditions effective for said test agent to modulate the biological activity of said polypeptide, and determining whether said test compound modulates said biological activity.

Contacting the polynucleotide or polypeptide with the test compound can be accomplished by any suitable method and/or means that places the compound in a position to functionally control expression or biological activity of the gene or its product in the sample. Functional control indicates that the compound can exert its physiological effect through whatever mechanism it works. The choice of the method and/or means can depend upon the nature of the compound and the condition and type of environment in which the gene or its product is presented, e.g., lysate, isolated, or in a cell population (such as, in vivo, in vitro, organ explants, etc.). For example, if the cell population is an in vitro cell culture, the compound can be contacted with the cells by adding it directly into the culture medium. If the compound cannot dissolve readily in an aqueous medium, it can be incorporated into liposomes, or another lipophilic carrier, and then administered to the cell culture. Contact can also be facilitated by incorporation of compound with carriers and delivery molecules and complexes, by injection, by infusion, etc.

After the agent has been administered in such a way that it can gain access to the gene or gene product (e.g., MSI-H marker, including DNA, mRNA, and polypeptides), it can be determined whether the test compound modulates its expression or biological activity. Modulation can be of any type, quality, or quantity, e.g., increase, facilitate, enhance, up- regulate, stimulate, activate, amplify, augment, induce, decrease, down-regulate, diminish, lessen, reduce, etc. The modulatory quantity can also encompass any value, e.g., 1%, 5%, 10%, 50%, 75%, 1-fold, 2-fold, 5-fold, 10-fold, 100-fold, etc. To modulate gene expression means, e.g., that the test compound has-an effect on its expression, e.g., to effect the amount of transcription, to effect RNA splicing, to effect translation of the RNA into polypeptide, to effect RNA or polypeptide stability, to effect polyadenylation or other processing of the RNA, to effect post-transcriptional or post- translational processing, etc. To modulate biological activity means, e.g., that a functional activity of the polypeptide is changed in comparison to its normal activity in the absence of the compound. This effect includes, increase, decrease, block, inhibit, enhance, etc.

A test compound can be of any molecular composition, e.g., chemical compounds, biomolecules, such as polypeptides, lipids, nucleic acids (e.g., antisense to a polynucleotide) carbohydrates, antibodies, ribozymes, double-stranded RNA, aptamers, etc. For example, if a MSI-H marker polypeptide to be modulated is a cell-surface molecule, a test compound can be an antibody that specifically recognizes it and, e.g., causes the polypeptide to be internalized, leading to its down regulation on the surface of the cell. Such effect does not have to be permanent, but can require the presence of the antibody to continue the down- regulatory effect. Antibodies can also be used to modulate the biological activity of a polypeptide in a lysate or other cell-free form.

Drug Screening

Several in vivo methods can be used to identify compounds that modulate expression of MSI-H marker nucleic acids (SEQ ID Nos: 1-34, or a sequence complementary thereto) and/or alter for example, inhibit the bioactivity of the encoded polypeptide (e.g., SEQ ID Nos: 35-68).

Drug screening is performed by adding a test compound to a sample of cells, and monitoring the effect. A parallel sample which does not receive the test compound is also monitored as a control. The treated and untreated cells are then compared by any suitable phenotypic criteria, including but not limited to microscopic analysis, viability testing, ability to replicate, histological examination, the level of a particular RNA or polypeptide associated with the cells, the level of enzymatic activity expressed by the cells or cell lysates, and the ability of the cells to interact with other cells or compounds. Differences between treated and untreated cells indicates effects attributable to the test compound.

Desirable effects of a test compound include an effect on any phenotype that was conferred by the cancer-associated marker nucleic acid sequence. Examples include a test compound that limits the overabundance of mRNA, limits production of the encoded protein, or limits the functional effect of the protein. The effect of the test compound would be apparent when comparing results between treated and untreated cells.

The invention thus also encompasses methods of screening for agents which inhibit expression of MSI-H marker nucleic acid (SEQ ID Nos: 1-34, or a sequence complementary thereto) in vitro, comprising exposing either a cell or tissue in which MSI-H marker nucleic acid mRNA is detectable or cultured cells comprising and capable of expressing MSI-H marker nucleic acid to an agent in order to determine whether the agent is capable of inhibiting production of the mRNA; and determining the level of mRNA in the exposed cells or tissue, wherein a decrease in the level of the mRNA after exposure of the cell line to the agent is indicative of inhibition of the marker nucleic acid mRNA production.

Alternatively, the screening method may include in vitro screening of a cell or tissue in which MSI-H marker nucleic acid is overexpressed or polypeptide is detectable, or cultured cells which express MSI-H markers, or in which MSI-H marker nucleic acids are overexpressed, to an agent suspected of inhibiting expression of MSI-H marker nucleic acid and/or production of MSI-H marker protein; and determining the level of the MSI-H marker polynucleotide or polypeptide in the cells or tissue, wherein a decrease in the level of marker polynucleotide or polypeptide after exposure of the cells or tissue to the agent is indicative of inhibition of marker protein production or marker polypeptide expression.

The invention also encompasses in vivo methods of screening for agents which inhibit expression of the marker nucleic acids, comprising exposing a mammal having tumor cells or serum in which MSI-H marker mRNA or protein is detectable to an agent suspected of inhibiting production of marker mRNA or protein; and determining the level of marker mRNA or protein in serum or tumor cells of the exposed mammal. A decrease in the level of marker mRNA or protein after exposure of the mammal to the agent is indicative of inhibition of marker nucleic acid expression. Optionally, the effect of the candidate agent on the expression of at least one additional colorectal cancer associated marker may also be determined.

Accordingly, the invention provides a method comprising incubating a cell expressing the marker nucleic acids (SEQ ID Nos: 1-34, or a sequence complementary thereto) with a test compound and measuring the DNA, mRNA, or protein level. The invention further provides a method for quantitatively determining the level of expression of the marker nucleic acids in a cell population or biological sample, and a method for determining whether an agent is capable of increasing or decreasing the level of expression of the MSI-H marker nucleic acid in a cell population or biological sample. In one embodiment, a method for determining whether an agent is capable of increasing or decreasing the level of expression of MSI-H marker nucleic acid in a cell population comprises the steps of (a) preparing cell extracts from control and agent-treated cell populations, (b) isolating MSI-H marker polypeptide from the cell extracts, (c) quantifying (e.g., in parallel) the amount of an immunocomplex formed between MSI-H marker polypeptide and an antibody specific to said polypeptide. The MSI-H marker polypeptide of this invention may also be quantified by assaying for its bioactivity. Agents that induce an increase in MSI-H marker nucleic acid expression may be identified by their ability to increase the amount of immunocomplex formed in the treated cell as compared with the amount of the immunocomplex formed in the control cell. In a similar manner, agents that decrease expression of MSI-H marker nucleic acid may be identified by their ability to decrease the amount of the immunocomplex formed in the treated cell extract as compared to the control cell. mRNA levels can be determined by Northern blot hybridization. mRNA levels can also be determined by methods involving PCR. Other sensitive methods for measuring mRNA, which can be used in high throughput assays, e.g., a method using a DELFIA endpoint detection and quantification method, are described, e.g., in Webb and Hurskainen (1996) Journal of Biomolecular Screening 1:119. MSI-H marker protein levels can be determined by immunoprecipitations or immunohistochemistiy using an antibody that specifically recognizes the protein product of SEQ ID Nos: 35-68.

Agents that are identified as active in the drug screening assay are candidates to be tested for their capacity to block cell proliferation activity. These agents would be useful for treating a disorder involving aberrant growth of cells, especially colon cells, especially colorectal cancer.

A variety of assay formats will suffice and, in light of the present disclosure, those not expressly described herein will nevertheless be comprehended by one of ordinary skill in the art. For instance, the assay can be generated in many different formats, and include assays based on cell-free systems, e.g., purified proteins or cell lysates, as well as cell-based assays which utilize intact cells.

In many drug screening programs which test libraries of compounds and natural extracts, high throughput assays are desirable in order to maximize the number of compounds surveyed in a given period of time. Assays of the present invention which are performed in cell-free systems, such as may be derived with purified or semi-purified proteins or with lysates, or with proteins purified or semi-purified from serum, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

### EXAMPLES

### Example 1

### Identification of Differentially Expressed Sequences

Thirty-four pairs of well-characterized, microdissected samples of colorectal cancer tissue and matched normal adjacent tissues were obtained from consenting patients. The location in the colon, stage of disease, and microsatellite status were determined for each sample. Total RNA was extracted from the samples using RNeasy kits (QIAGEN, Valencia, Calif.) according to the manufacturer's instructions. Each purified RNA sample was visually inspected for quality on an agarose gel, and quantified using the RiboGreen® assay (Molecular Probes, Eugene, Oreg.).

To identify differentially expressed genes, expression profiling was performed on custom, high-density nylon membrane arrays. Arrays were generated by spotting sequence- verified PCR products onto Nytran® SuperCharge nylon membranes (Schleicher & Schuell BioScience, Inc., Keene, N.H.) using a BioGrid robotic arrayer (Genomic Solutions Inc., Ann Arbor, Mich.). These arrays contain over 13,000 clones, which represent ˜5400 distinct genes.

Due to the limited amount of RNA isolated from the microdissected samples, one microgram of total RNA from each sample was amplified using SMART™ technology (BD Biosciences/Clontech, Palo Alto, Calif.), which maintains the original representation of the RNA sample. Complex ³³P-labeled probes were prepared from the amplified cDNA. Probes from matched tumor and normal tissue were hybridized to identical nylon membranes. Signals were captured via a Storm™ Phosphorimager (Amersham Biosciences, Piscataway, N.J.) and analyzed via ArrayVision™ (Imaging Research Inc., St. Catharines, Ontario, Canada) and GeneSpring software (Silicon Genetics, Redwood City, Calif.).

Expression data were normalized and scaled according to the GeneSpring default parameters. For each array, the signal of each spot was divided by the average signal of all the spots on the particular array. Next, the signal for each gene was divided by the median signal of that gene from all measurements. Finally, the signals from each cancer sample were divided by the signals from the matched normal sample to generate the ratio data for each tumor normal pair. Data represented in this manner indicate the fold overexpression (or underexpression) for each gene on the array. Values greater than 1.0 represent genes that are overexpressed in the tumor tissue relative to the normal tissue, while values less than 1.0 indicated those genes that are underexpressed.

To identify genes that were overexpressed in the MSI-H tumors, a series of filters was used to reduce the data set. Starting with the entire normalized data set, both parametric and non-parametric tests were used to define genes that were statistically associated with either the MSI-H tumors or the MSS tumors. Next, for each of these two sets, a filter was used to set a high threshold for a minimum expression level in the MSI-H tumors. Specifically, this filter selected those genes in the MSI-H tumors that were at least 7 fold overexpressed in any one of the 15 tumor/normal pairs. Finally, a Venn diagram was utilized to determine the distinct genes from these two similarly derived sets. This analysis resulted in 34 genes that were overexpressed in the MSI-H tumors, but not in the MSS tumors. These genes are identified in Table 1.

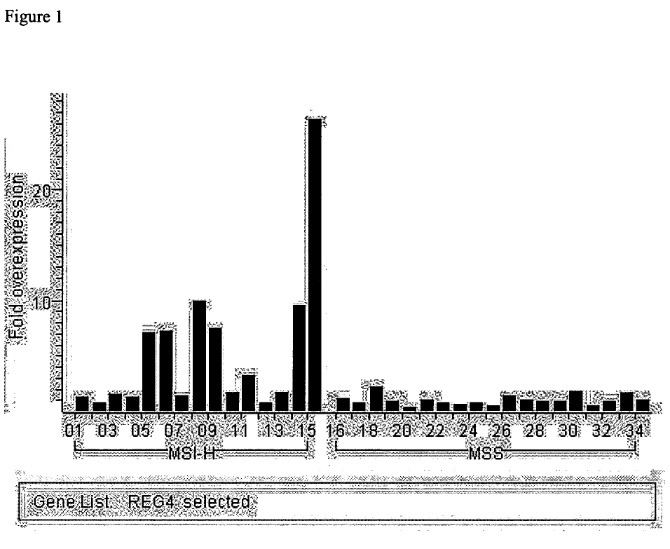

An example of a differentially expressed gene, REG4, is shown in FIG. 1. In this graph, the 15 MSI-H tumors are grouped on the left side of the x-axis, and the 19 MSS tumors are on the right. The y-axis represents the fold overexpression of REG4 in each of the matched tumor/normal pairs. REG4 was greater than 2 fold overexpressed in 7 of the 15 MSI-H tumors, but was not significantly overexpressed in any of the 19 MSS tumors. The average fold overexpression of REG4 was 5.4 in the MSI-H tumors, and 1.0 in the MSS tumors.

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the following claims.

1. A method of detecting microsatellite instability in an individual comprising:
measuring the expression of the sequence of one or more of SEQ ID Nos 1-34 in said individual, wherein microsatellite instability is detected where said sequence is overexpressed by at least 2-fold compared to the expression of said sequence in an individual known to not have microsatellite instability.

2. The method of claim 1, wherein the step of measuring comprises:
(a) obtaining a colorectal tissue sample from said individual;
(b) contacting said sample with a nucleic acid probe which is capable of hybridizing under stringent hybridization conditions to said sequence of one or more of SEQ ID Nos 1-34;
(c) detecting hybridization of said nucleic acid probe to said sequence of one or more of SEQ ID Nos 1-34.

3. The method of claim 1, wherein said individual is a human.

4. The method of claim 2, wherein said nucleic acid probe is detectably labeled.

5. A method of identifying colon tissue having microsatellite instability, comprising:
(a) obtaining a colon tissue sample from an individual;
(b) detecting the expression of one or more of the sequences of SEQ ID Nos 1-34, wherein colon tissue having microsatellite instability is detected if the expression of said one or more of the sequences of SEQ ID Nos 1-34 is at least 2-fold greater than that of the same sequence in a colon tissue sample known to not have microsatellite instability.

6. The method of claim 5, wherein said step of detecting comprises:
(a) contacting said colon tissue sample with a nucleic acid probe which is capable of hybridizing under stringent hybridization conditions to said one or more of the sequences of SEQ ID Nos 1-34; and
(b) detecting hybridization of said nucleic acid probe to said one or more sequences, wherein detection of hybridization is indicative of the expression of said one or more of the sequences of SEQ ID Nos 1-34.

7. The method of claim 6, wherein said nucleic acid probe is detectably labeled.

8. The method of claim 5, wherein said individual is a human.

9. The method of claim 5, wherein said colon tissue sample is a tumor sample.

10. The method of claim 9, wherein said tumor is a malignant tumor.

11. A method of monitoring the onset, progression, or regression of cancer or a pre- malignant condition thereof in a subject, the method comprising:
(a) detecting in a biological sample of the subject at a first point in time, the expression of one or more nucleic acid sequences comprising one or more nucleic acid sequences selected from the group consisting of SEQ ID NOs: 1-34;
(b) repeating step (a) at a subsequent point in time; and
(c) comparing the expression level detected in steps (a) and (b), wherein a change in the expression level is indicative of progression of cancer or a pre-malignant condition thereof in the subject.

12. The method of claim 11, wherein the change in the expression level is either an increase or decrease.

13. The method of claim 12, wherein said increase or decrease is an increase or decrease of at least 2 fold.

14. A method of determining prognosis for cancer or a pre-malignant condition thereof in a subject, comprising:
(a) detecting in a biological sample of the subject, the expression level of one or more nucleic acid sequences comprising one or more nucleic acid sequences selected from the group consisting of SEQ ID NOs: 1-34;
(b) comparing the expression level detected in steps (a) with a reference expression level of said nucleic acid sequences; and
(c) evaluating the prognosis of the subject based on the comparison in step (b).

15. The method of claim 14, wherein the reference expression level is the expression level of said nucleic acid sequences in cancer free or normal sample.

16. The method of claim 14, wherein the reference expression level is the expression level of said nucleic acid sequences in cancer samples that are known not to progress to aggressive form.

17. The method of claim 14, wherein the reference expression level is the expression level of said nucleic acid sequence in a MSS colon tumor.

18. A method of determining the efficacy of a test compound for inhibiting cancer in a subject, the method comprising comparing a) the expression level of one or more nucleic acid sequences in a first biological sample from the subject wherein the sample has been exposed to the test compound, with b) the expression level of said nucleic acid sequences in a second biological sample from the subject wherein the sample has not been exposed to the test compound, said nucleic acid sequences comprising one or more nucleic acid sequences selected from the group consisting of SEQ ID NOs: 1-34, wherein a change of at least two fold in the expression level of said nucleic acid sequences is an indication that the test compound is efficacious for inhibiting cancer in the subject.

19. The method of claim 18, wherein the change in the expression level is either an increase or decrease.

20. A method of determining the efficacy of a therapy for inhibiting cancer in a subject, the method comprising comparing a) the expression level of one or more nucleic acid sequences in a first biological sample from the subject prior to providing at least a portion of the therapy to the subject, with b) the expression level of said nucleic acid sequences in a second biological sample from the subject following the provision of at least a portion of the therapy, said nucleic acid sequences comprising one or more nucleic acid sequences selected from the group consisting of SEQ ID NOs: 1-34, wherein a change of at least two fold in the expression level of said nucleic acid sequences is an indication that the therapy is efficacious for inhibiting cancer in the subject.

21. The method of claim 20, wherein the change in the expression level is either an increase or decrease.

22. A method of selecting a composition for inhibiting cancer in a subject, the method comprising:
(a) obtaining a first biological sample comprising cancer cells from the subject;
(b) separately exposing aliquots of the sample in the presence of a plurality of test compositions;
(c) comparing the expression level of one or more nucleic acid sequences in each of the aliquots from (b) with the expression level in the sample produced by (a), said nucleic acid sequences comprising one or more nucleic acid sequences selected from the group consisting of SEQ ID NOs: 1-34; and
(d) selecting one of the test compositions which induces a change of at least two fold in the expression level of said nucleic acid sequences in one aliquot containing the test composition.

23. The method of claim 22, wherein the change in the expression level is either an increase or decrease.

24. A method of monitoring the onset, progression, or regression of cancer in a subject, comprising:
(a) contacting at a first point in time a first biological sample with one or more polypeptide ligands that specifically bind to one or more polypeptides comprising a polypeptide sequence selected from the group consisting of SEQ ID NOs: 35-68, determining specific binding between the polypeptide ligands and the polypeptides;
(b) contacting at a subsequent point in time a second biological sample with said polypeptide ligands that specifically bind to one or more polypeptides comprising a polypeptide sequence selected from the group consisting of SEQ ID NOs: 35-68, determining specific binding between the polypeptide ligands and the polypeptides; and
(c) comparing the specific binding in the first biological sample to the specific binding in the second biological sample, wherein a significant change in the specific binding is an indication of the onset, progression, or regression of cancer.

25. A method of determining prognosis for cancer or a pre-malignant condition thereof in a subject, comprising:
(a) contacting a biological sample obtained from a subject having cancer with one or more polypeptide ligands that bind specifically to one or more polypeptides comprising a polypeptide sequence selected from the group consisting of SEQ ID NOs: 35-68;
(b) determining specific binding;
(c) comparing the specific binding between the polypeptide ligands and the polypeptides in the sample with the specific binding between the polypeptide ligands and the polypeptides either in a cancer-free sample or in a cancer sample that is known not to progress to aggressive form; and
(d) evaluating the prognosis of the subject based on the comparison in step (c).

26. A method of determining the efficacy of a test compound for inhibiting cancer in a subject, the method comprising comparing a) in a first biological sample from the subject binding between one or more polypeptide ligands that specifically bind to one or more polypeptides comprising a polypeptide sequence selected from the group consisting of SEQ ID NOs: 35-68 and one or more polypeptides comprising a polypeptide sequence selected from the group consisting of SEQ ID NOs: 35-68, wherein the sample has not been exposed to the test compound, with b) in a second biological sample from the subject, the specific binding of said polypeptide ligands and said polypeptides, wherein the sample has been exposed to the test compound, and wherein a significant change in the specific binding is an indication that the test compound is efficacious for inhibiting cancer in the subject.

27. A method of determining the efficacy of a therapy for inhibiting cancer in a subject, comprising comparing a) in a first biological sample from the subject prior to a treatment, binding between one or more polypeptide ligands that specifically bind to one or more polypeptides comprising a polypeptide sequence selected from the group consisting of SEQ ID NOs: 35-68 and one or more polypeptides comprising a polypeptide sequence selected from the group consisting of SEQ ID NOs: 35-68, with b) in a second biological sample from the subject following the treatment, the specific binding of said polypeptide ligands and said polypeptides, and wherein a significant change in the specific binding is an indication that the test compound is efficac for inhibiting cancer in the subject.
